SEQUENCE LISTING (5 sequences)

SEQ ID NO: 1 - DNA, 20 bp, Artificial Sequence
  oligonucleotide AR369 - synthetic tracrRNA bearing 3′ DNA extension
  misc_feature (20)..(20): 3′ iSp18/[tracrRNA]
tacatttaagaccctaatat

SEQ ID NO: 2 - DNA, 20 bp, Artificial Sequence
  synthetic crRNA
cttcgcggcagatataatgg

SEQ ID NO: 3 - DNA, 20 bp, Artificial Sequence
  synthetic crRNA
ccgaccacgccagcatatcg

SEQ ID NO: 4 - DNA, 66 bp, Artificial Sequence
  oligonucleotide EN45
  misc_feature (1)..(1): 5′ DBCO-TEG
gcttgggtgtttaaccgttttcgcatttatcgtgaaacgctttcgcgtttttcgtgcgccgcttca

SEQ ID NO: 5 - DNA, 84 bp, Artificial Sequence
  oligonucleotide EN47 - MuA bottom strand bearing 3′ DNA extension annealing to the tracrRNA
gatctgaagcggcgcacgaaaaacgcgaaagcgtttcacgataaatgcgaaaacttttttttttatattagggtcttaaatgta

(19) United States
(12) Patent Application Publication
(10) Pub. No.: US 2022/0403368 A1
(43) Pub. Date: Dec. 22, 2022

(54) METHODS AND SYSTEMS FOR PREPARING A NUCLEIC ACID CONSTRUCT FOR SINGLE MOLECULE CHARACTERISATION

(71) Applicant: Oxford Nanopore Technologies PLC, Oxford (GB)

(72) Inventors: Rebecca Victoria Bowen, Oxford (GB); Etienne Raimondeau, Oxford (GB); James Edward Graham, Oxford (GB); James White, Oxford (GB)

(73) Assignee: Oxford Nanopore Technologies PLC, Oxford (GB)

(21) Appl. No.: 17/763,716

(22) PCT Filed: Sep. 25, 2020
(86) PCT No.: PCT/GB2020/052331

§ 371 Date: Mar. 25, 2022

(30) Foreign Application Priority Data
Sep. 27, 2019 (GB) 1913997.1

(51) Int. Cl. C12N 15/10

(52) U.S. Cl. CPC C12N 15/102; C12N 2310/20

(57) ABSTRACT

A method of preparing a nucleic acid construct for single molecule characterisation, comprising contacting a target polynucleotide with: a polynucleotide-guided effector protein, a guide polynucleotide; a transposase; and a transposable element comprising a modified polynucleotide, wherein the polynucleotide-guided effector protein directs said transposase to a region of interest within the target polynucleotide and the transposase inserts the transposable element into the polynucleotide, thereby producing a nucleic acid construct for single molecule characterisation.

### FIELD OF THE INVENTION

The present invention relates to methods and systems for preparing a nucleic acid construct for single molecule characterisation.

### BACKGROUND

There is currently a need for rapid and cheap polynucleotide (e.g. DNA or RNA) sequencing and identification technologies across a wide range of applications. Existing technologies are slow and expensive mainly because they rely on amplification techniques to produce large volumes of polynucleotide and require a high quantity of specialist fluorescent chemicals for signal detection.

Transmembrane pores (nanopores) have great potential as direct, electrical biosensors for polymers and a variety of small molecules. In particular, recent focus has been given to nanopores as a potential DNA sequencing technology.

When a potential is applied across a nanopore, there is a change in the current flow when an analyte, such as a nucleotide, resides transiently in the barrel for a certain period of time. Nanopore detection of the nucleotide gives a current change of known signature and duration. In the strand sequencing method, a single polynucleotide strand is passed through the pore and the identity of the nucleotides are derived. Strand sequencing can involve the use of a molecular brake to control the movement of the polynucleotide through the pore.

There are many commercial situations, including polynucleotide sequencing and identification technologies, which require the preparation of a nucleic acid library. This may be achieved using a transposase.

### SUMMARY OF THE INVENTION

The present inventors have identified a novel method for preparing a nucleic acid constructs for single molecule characterization, for example by nanopore sequencing. In the method, a polynucleotide-guided effector protein (PGEP) is used to direct a transposase to a region of interest within a target polynucleotide. Upon such direction, the transposase interacts with a transposable element that comprises a polynucleotide, such as a modified polynucleotide, to mediate insertion of the substrate to the region of interest. In this way, a modified polynucleotide beneficial to single molecule characterization may be introduced to a target polynucleotide to facilitate its characterization.

The present inventors have demonstrated that the PGEP and the transposase may interact in a variety of ways, and moreover, that this interaction can be manipulated in order to tune the activity of the transposase. In this way, the accuracy of insertion can be improved, and off-target transposase activity minimized.

Accordingly, provided herein is a method of preparing a nucleic acid construct for single molecule characterisation, comprising contacting a target polynucleotide with:
a polynucleotide-guided effector protein,
a guide polynucleotide;
a transposase; and
a transposable element comprising a modified polynucleotide,
   wherein the polynucleotide-guided effector protein directs said transposase to a region of interest within the target polynucleotide and the transposase inserts the transposable element into the polynucleotide, thereby producing a nucleic acid construct for single molecule characterisation.

Also provided is a method of preparing a nucleic acid construct, comprising contacting a target polynucleotide with:
a polynucleotide-guided effector protein
a guide polynucleotide;
a transposase; and
a transposable element,
   wherein the polynucleotide-guided effector protein and transposase are genetically fused or connected via a linker moiety such that the transposase is directed to a region of interest within the target polynucleotide and inserts the transposable element into the target polynucleotide, thereby preparing a nucleic acid construct.

Also provided is a system for preparing a nucleic acid construct for single molecule characterisation, comprising:
a polynucleotide-guided effector protein;
a guide polynucleotide;
a transposase; and
a transposable element comprising a modified polynucleotide,
   wherein the polynucleotide-guided effector protein directs said transposase to a region of interest within the target polynucleotide and further wherein the transposase inserts the transposable element into the polynucleotide, thereby producing a nucleic acid construct for single molecule characterisation.

Also provided is a system for preparing a nucleic acid construct, comprising:

a polynucleotide-guided effector protein;

a guide polynucleotide;

a transposase; and

a transposable element,

wherein the polynucleotide-guided effector protein and transposase are genetically fused or connected via a linker moiety, such that the transposase is directed to a region of interest within the target polynucleotide and inserts the transposable element into the target polynucleotide, thereby preparing a nucleic acid construct.

Also provided is a method of detecting and/or characterising a target polynucleotide in a sample, comprising:
(i) preparing a nucleic acid construct for single molecule characterisation according to the method of any one of claims 1 to 27;
(ii) contacting the nucleic acid construct with a membrane comprising a transmembrane pore;
(iii) applying a potential difference across the membrane; and
(iv) taking one or more measurements resulting from the contacting of the nucleic acid construct with the pore thereby detecting and/or characterising the target polynucleotide to determine the presence or absence of the target polynucleotide and/or one or more characteristics of the target polynucleotide.

### DESCRIPTION OF THE FIGURES

It is to be understood that Figures are for the purpose of illustrating particular embodiments of the invention only, and are not intended to be limiting.

FIG. 1 shows schematically how a Cas9 enzyme A, with bound tracrRNA B and crRNA C, may be used to bind a target dsDNA molecule D containing a protospacer-adjacent motif (PAM) E and a Cas9 bound or linked transposon protein complex may insert a dsDNA tag in the dsDNA molecule. Cas9 can be linked to one or multiple transposon proteins such as MuA or Tn5 F. In this example, the linkage G between Cas9 and the transposon protein is occurring between Cas9 tracrRNA B and one of the transposon tag strands H. Such linkage is also possible between the Cas9 crRNA C and the transposon tag strands H. The transposon tag strands J and H can contain a 5′DBCO group used for clicking sequencing adapter. The tracrRNA and crRNA may be incorporated as a single-guide RNA (sgRNA) molecule by interlinking the two with a hairpin. The transposon proteins insert the top and bottom transposon strands into the molecule thus effectively cleaving the molecule using two tagged dsDNA fragments, K and L, both bearing transposon tag strands.

FIG. 2 shows schematically how a Cas9 enzyme A, with bound tracrRNA B and crRNA C, may be used to bind a target dsDNA molecule D containing a protospacer-adjacent motif (PAM) E and a Cas9 linked transposon protein complex may insert a dsDNA cargo in the dsDNA molecule. In this example, Cas9 is fused to one of multiple transposon proteins F via a protein linker H. The transposon is bound to a dsDNA cargo consisting of a insert sequence J and two adapter sequences K. The linkage between Cas9 and the transposon proteins is also possible between the Cas9 crRNA C or tracrRNA and the transposon tag strands K. The tracrRNA and crRNA may be incorporated as a single-guide RNA (sgRNA) molecule by interlinking the two with a hairpin. The transposon proteins insert the dsDNA cargo into the dsDNA molecule forming the molecule M which can be used for downstream applications such as those described in FIG. 18.

FIG. 3 shows schematically how a Cas12k enzyme A, with bound tracrRNA B and crRNA C, may be used to bind a target dsDNA molecule D containing a protospacer-adjacent motif (PAM) E. Cas12k binds the transposon protein TniQ F which in turn binds to the transposon proteins H and G. The transposon is bound to a dsDNA cargo consisting of a insert sequence J and two adapter sequences upstream (LE site) K and downstream (RE site) L of the insert sequence. The tracrRNA and crRNA may be incorporated as a single-guide RNA (sgRNA) molecule by interlinking the two with a hairpin. The transposon proteins insert the dsDNA cargo into the molecule thus forming the molecule M which can be used for downstream applications such as those described in FIG. 18.

FIG. 4 shows schematically how a Cas12k enzyme A, with bound tracrRNA B and crRNA C, may be used to bind a target dsDNA molecule D containing a protospacer-adjacent motif (PAM) E. Cas12k binds the transposon protein TniQ F which in turn binds to the transposon proteins H and G. The transposon is bound to a dsDNA cargo consisting of a insert sequence J with a single stranded section and two adapter sequences upstream (LE site) K and downstream (RE site) L of the insert sequence. The tracrRNA and crRNA may be incorporated as a single-guide RNA (sgRNA) molecule by interlinking the two with a hairpin. The transposon proteins insert the dsDNA cargo into the molecule thus forming the molecule M which can be used for downstream applications such as those described in FIG. 18.

FIG. 5 shows schematically how a Cas12k enzyme A, with bound tracrRNA B and crRNA C, may be used to bind a target dsDNA molecule D containing a protospacer-adjacent motif (PAM) E. Cas12k binds the transposon protein TniQ F which in turn binds to the transposon proteins H and G. The transposon is bound to a dsDNA cargo consisting of a insert sequence J containing a double strand break and two adapter sequences upstream (LE site) K and downstream (RE site) L of the insert sequence. The tracrRNA and crRNA may be incorporated as a single-guide RNA (sgRNA) molecule by interlinking the two with a hairpin. The transposon proteins insert the dsDNA cargo into the molecule thus forming the molecule M which can be used for downstream applications such as those described in the FIGS. 23, 26, 34, 35 and 36.

FIG. 6 shows schematically how a Cascade complex formed by Cas8 A, Cas7 B and Cas6 C, with bound crRNA D, may be used to bind a target dsDNA molecule E containing a protospacer-adjacent motif (PAM) F. The cascade complex binds the transposon protein TniQ G which in turn binds to the transposon complex formed by proteins H, J and K. The transposon is bound to a dsDNA cargo consisting of a insert sequence L and two adapter sequences upstream M and downstream N of the insert sequence. The tracrRNA and crRNA may be incorporated as a single-guide RNA (sgRNA) molecule by interlinking the two with a hairpin. The transposon proteins insert the dsDNA cargo into the molecule thus forming the molecule O which can be used for downstream applications as those described in the FIG. 18.

FIG. 7 shows schematically how a Cascade complex formed by Cas8 A, Cas7 B and Cas6 C, with bound crRNA D, may be used to bind a target dsDNA molecule E containing a protospacer-adjacent motif (PAM) F. The cascade complex binds the transposon protein TniQ G which in turn binds to the transposon complex formed by proteins H, J and K. The transposon is bound to a dsDNA cargo consisting of a insert sequence L containing a single stranded section and two adapter sequences upstream M and downstream N of the insert sequence. The tracrRNA and crRNA may be incorporated as a single-guide RNA (sgRNA) molecule by interlinking the two with a hairpin. The transposon proteins insert the dsDNA cargo into the molecule thus forming the molecule O which can be used for downstream applications as those described in the FIG. 18.

FIG. 8 shows schematically how a Cascade complex formed by Cas8 A, Cas? B and Cas6 C, with bound crRNA D, may be used to bind a target dsDNA molecule E containing a protospacer-adjacent motif (PAM) F. The Cascade complex binds the transposon protein TniQ G which in turn binds to the transposon complex formed by proteins H, J and K. The transposon is bound to a dsDNA cargo consisting of a insert sequence L containing a double strand break and two adapter sequences upstream M and downstream N of the insert sequence. The tracrRNA and crRNA may be incorporated as a single-guide RNA (sgRNA) molecule by interlinking the two with a hairpin. The transposon proteins insert the dsDNA cargo into the molecule thus forming the molecule O which can be used for downstream applications as those described in the FIGS. 23, 26, 34, 35 and 36.

FIG. 9 shows one possible workflow by which a target DNA molecule may be sequenced by inserting clickable tags via transposon proteins bound or linked to CRISPR/Cas, clicking sequencing adapters to the tags, and introducing into a sequencing device. A mixture of target (A) and non-target (B) high-molecular weight DNA is mixed with CRISPR-Transposon RNPs C. Upon the RNPs binding to the target DNA, a double-strand break is introduced that cleaves the target molecule into two fragments D and E and attaching tag sequences with a 5′ DBCO group to both fragments. Upon removal of bound RNPs, sequencing adapters containing a 3′ azide group are attached to the fragments. This yields two adapter-ligated target fragments F and G, which when introduced into a nanopore sequencing flowcell comprising membrane H and pore J, may both be sequenced. Both target and non-target molecules are introduced into the flowcell, but only target molecules tether onto the membrane and are sequenced.

FIG. 10 shows one possible workflow by which a target DNA molecule may be sequenced by inserting clickable tags via CRISPR/Cas - transposon proteins, clicking sequencing adapters to the cargo, and introducing into a sequencing device. A mixture of target (A) and non-target (B) high-molecular weight DNA is mixed with CRISPR-Transposon RNPs C. Upon the RNPs binding to the target DNA, the cargo DNA bound to the transposon proteins is introduced into the target molecule D. The cargo DNA sequence contains exposed 5′ DBCO tags. Upon removal of bound RNPs, sequencing adapters containing a 3′ azide group are attached to the fragments tags in the target molecule E. This yields one adapter-ligated target fragment F, which when introduced into a nanopore sequencing flowcell comprising membrane G and pore H, may be sequenced. Both target and non-target molecules are introduced into the flowcell, but only target molecules tether onto the membrane and are sequenced.

FIG. 11 shows one possible workflow by which a target DNA molecule may be amplified and sequenced by inserting primer binding site via CRISPR/Cas-transposon proteins, clicking sequencing adapters to the amplified product, and introducing into a sequencing device. A mixture of target (A) and non-target (B) high-molecular weight DNA is mixed with CRISPR-Transposon RNPs C. Upon the RNPs binding to the target DNA, the cargo DNA bound to the transposon proteins is introduced into the target molecule D. The cargo DNA sequence contains exposed sequences which can bind to specific primers. Upon removal of bound RNPs, specific primers containing a 5′DBCO group are used to amplify the target molecule E forming the molecule F. Sequencing adapters containing a 3′ azide group are attached to the fragments tags in the target molecule F. This yields one adapter-ligated target fragment G, which when introduced into a nanopore sequencing flowcell comprising membrane H and pore J, may be sequenced. Both target and non-target molecules are introduced into the flowcell, but only target molecules tether onto the membrane and are sequenced.

FIG. 12 shows one possible workflow by which a target DNA molecule may be sequenced by attaching tags via transposons bound to CRISPR/Cas RNPs, clicking to sequencing adapters, and introducing into a sequencing device. In tube B, crRNAs are annealed to tracrRNA and RNPs are formed by incubating this mixture with Cas9 for 10 mins at room temperature. Subsequently, the content of tube B is added to tube A containing high molecular weight DNA and the CRISPR/Cas RNPs are allowed to bind to the DNA for 15-30mins at 37° C. The Transposon RNPs are added to the mixture and incubated for 15-30 mins at 37° C. to allow cleavage and tagging of the target DNA. Following optional SPRI purification of the mixture, the fragments of interest are clicked to the sequencing adaptor using click chemistry forming the sequencing library. The sample is introduced to the sequencing device.

FIG. 13 shows one possible workflow by which a target DNA molecule may be sequenced by attaching tags via transposons bound to CRISPR/Cas RNPs, clicking to sequencing adapters, and introducing into a sequencing device. In tube B, crRNAs are annealed to tracrRNA and the transposon tag strands are assembled and RNPs are formed by incubating this mixture with Cas9 and the transposon proteins for 30 mins at room temperature. Subsequently, the content of tube B is added to tube A containing high molecular weight DNA and the CRISPR Transposon RNPs are allowed to bind to the DNA for 15-30mins at 37° C. to allow cleavage and tagging of the target DNA. Following optional SPRI purification of the mixture, the fragments of interest are clicked to the sequencing adaptor using click chemistry forming the sequencing library. The sample is introduced to the sequencing device.

FIG. 14 shows one possible workflow by which a target DNA molecule may be sequenced by inserting a cargo containing tags via CRISPR Transposon RNPs, clicking to sequencing adapters, and introducing into a sequencing device. In tube B, crRNAs are annealed to tracrRNA and and RNPs are formed by incubating this mixture with Cas12k and the transposon proteins (TniQ, TnsB and TnsC in this example) in presence of the cargo DNA for 30 mins at room temperature. Subsequently, the content of tube B is added to tube A containing high molecular weight DNA and the CRISPR Transposon RNPs are allowed to bind to the DNA for 15-30mins at 37° C. to allow cleavage and insertion of the cargo in the target DNA. Following optional SPRI purification of the mixture, the fragments of interest are clicked to the sequencing adaptor using click chemistry forming the sequencing library. The sample is introduced to the sequencing device.

FIG. 15 explores the sequencing pattern of a single dsDNA break in the region of interest (ROI) induced by CRISPR Transposon RNP (A). The two fragments (B and C) generated contains each a transposon tag D and E which are accessible for sequencing adaptor ligation. Fragment B is read in the antisense direction (-) and fragment C in the sense direction (+) resulting in a decreasing coverage depth (D) from the cut location in both direction.

FIG. 16 explores the sequencing pattern of a double dsDNA break in the flanking regions of the region of interest (ROI) induced by CRISPR Transposon RNP (A and B). The one (C) of the three fragments (C and D and E) generated contains a transposon tag F on each ends which are accessible for sequencing adaptor ligation. The Fragment C is read in the antisense direction (-) and in the sense direction (+) resulting in a even coverage depth (G) in both direction.

FIG. 17 compares the different approaches used for Cas9-MuA enrichment. Panel 1 shows an experiment in which MuA bound to dCas9 was used to introduce clickable tags to an 3.6kb analyte for sequencing analysis, in absence of MuA (subpanel A) or in absence of Cas9 (subpanel B), with 0 or 150mM of additional NaCl in the reaction (subpanels C and C(2)). The panel shows the start of the sequencing reads in the forward (blue) or reverse direction (magenta). The Panel 2 shows the pileup of the reads on the 3.6kb analyte for the same experiment. The binding site of each of the dCas9 proteins is marked by a vertical dotted line.

FIG. 18 shows how a Cas-transposase system may be used to insert a cargo bearing a tract of modified bases or sequence recognition motifs at a defined locus. A and B show possible cargo designs for the transposase system shown in FIG. 3, inserting a tract of modified bases or sequence recognition motifs respectively. C and D denote the left-end (LE) and right-end (RE) motifs of the transposase payload respectively. E denotes a modified base, which may be a biotinylated base, non-canonical base, abasic site, spacer, etc. F denotes the payload backbone, which may be of arbitrary sequence, length or structure (double-stranded or single-stranded in character). G denotes a sequence recognition element for a binding protein; for example, the lac or tet operator sequence, a restriction enzyme binding site, or binding site for an engineered zinc finger protein. H, I, J, K denote integration products; H and I are the products of cargo A, and J and K are the products of cargo B. L denotes the protospacer-adjacent motif (PAM). M denotes a protein that recognises for example a chemical moiety attached to a modified base. N denotes a protein that recognises a specific sequence motif.

FIG. 19 shows examples of end-derivitisation for the detection of transposase-inserted cargo by a nanopore. A, blunt double-stranded end (F); B, 5′ or 3′ overhang (G) (or both, i.e. forked or splayed duplex); C, sequencing adaptor (H) ligated to molecule of interest carrying single-stranded helicase or translocase; D, sequencing adaptor (I) ligated to molecule of interest carrying double-stranded helicase or translocase. All examples include cargo (here labelled E) from FIG. 18 (labelled in FIG. 18 as A). The cargo may or may not carry bound protein, per FIG. 18 (c.f. Figure JG1, H and I)

FIG. 20 shows nanopore detection methods for Cas-transposase-mediated cargo insertion, for the example of a cargo of modified bases with or without bound protein. A, double-stranded translocation through nanopore (D) that can accommodate duplex but not modified base and/or bound protein. B, double-stranded translocation through nanopore (E) that can accommodate duplex and modified base and/or bound protein. C, single-stranded translocation through nanopore (F) that can accommodate single-strand DNA but not modified base and/or bound protein. All of the above examples (A, B, or C) may be mediated by a nucleic acid helicase/translocase enzyme (G, H or I), or the translocation may be enzyme-free (mediated by voltage). In C, the pore and/or enzyme unwind double-stranded DNA. Arrows indicate direction of translocation of the nucleic acid through the nanopore.

FIG. 21 shows examples of transposase cargos that direct the insertion of a tethering moiety into target DNA that may increase the local concentration of an analyte at a surface or enable its purification from a complex background. A, cargo bearing internal modified base(s) B that enable either the direct (via a hydrophobic moiety, e.g. cholesterol) or indirect (via a binding protein) tethering of the protein to a bead or membrane surface. K, cargo bearing internal ssDNA flap (L) that permits the hybridisation of a tethering oligonucleotide to the cargo. C, a system in which example target molecule F bearing Cas-transposase-integrated cargo from (A), tethered indirectly to surface D via binding protein E. An example of E is streptavidin, which may be coupled directly to surface D or indirectly via a second biotin moiety, forming a sandwich structure. An alternative example of a coupling interaction is where E is a hydrophobic moiety, e.g. cholesterol and E also hydrophobic, for example a membrane surface. The tethering interaction may increase the local concentration of the target molecule F on the surface E, enabling selection (for example by purification or proximity) of the target from background molecules G. H, a similar system to C in which a tethering oligo I hybridizes to the flap provided by the integrated cargo, enabling tethering to the surface J. Examples of I and J are where I is a biotinylated oligonucleotide and J a streptavidin-coated bead surface, or where I is a cholesterol-modified oligonucleotide and J a membrane surface.

FIG. 22 shows an example of a Cas-transposase cargo that may be used to derivatise DNA internally at a defined site for enzyme-free capture by a nanopore. A, cargo with two ssDNA extensions (B). Integration of DNA strand C bearing PAM D yields integrant E. The ssDNA flap of E is then captured by nanopore F and the duplex DNA of E unwound and one of the two strands translocated. The arrow shows the direction of translocation.

FIG. 23 shows an example of a Cas-transposase cargo that may be used to derivatise DNA internally at a defined site for enzyme-free capture by a nanopore. A, cargo consisting of two separate duplexes (B). Integration of DNA strand C bearing PAM D yields integrants E and F bearing a double-strand break. Either double-strand end of E or F is then captured by nanopore G and the duplex DNA translocated through the nanopore. The arrow shows the direction of translocation.

FIG. 24 shows example cargos for inserting sequencing adaptors using a Cas-transposase system involving one or two sequencing motors per cargo. A shows how a sequencing adaptor B carrying sequencing motor may be attached to a Cas-transposase cargo C via an ssDNA flap D carrying for example a click chemistry group E, yielding product F that is read in the direction indicated. The sequencing adaptor may be attached to the cargo before or after integration into the target molecule. G, H, I, J show some possible alternative configurations: G: two flaps that permit overlapping reads over the integration site; H: two flaps that do not yield overlapping reads; I: a cargo consisting of two duplexes and two ssDNA flaps that permit the attachment of two sequencing adaptors but yield a double-strand break after integration; J: per I, but with only one ssDNA flap, attaching one sequencing adaptor on one side of a double-strand break. For G and H the sequencing motors are attached to the same molecule in the final product and are connected via the intervening duplex.

FIG. 25 shows example configurations for the integration of two Cas-transposase pairs into a target molecule to sequence a specific region of interest (ROI). Only the final integration products are shown. A shows the integration products of two Cas-transposase cargos E where the two protospacer-adjacent motifs (PAMs) are on opposite strands but the cargos are identical (in the same orientation). B shows a similar scheme in which the PAMs are in the same strand but the cargos (E and F) are in opposing orientations. Both schemes A and B permit the sequencing of a specific region of interest (ROI) on both strands; specifically A would allow this scheme with a single type of cargo (E) but two different guide RNAs. Schemes C and D show two further possible configurations: C shows an ‘inside-out’ scheme in which reads are directed away from the ROI. Scheme D shows one possible scheme in which two sequencing adapters may be attached to each cargo to permit multiple overlapping reads over and outwards from a ROI. Further schemes are possible and the above schemes are not intended to be exhaustive. Block arrows show the expected sequencing read direction.

FIG. 26 shows an example of an integration product from a cargo such as that shown in FIG. 24, I, generating a double-strand break at the site of integration.

FIG. 27 shows an example of a combinatorial scheme involving the integration of two distinct Cas-transposase cargos (A, B) at two distinct loci (C, D). Integration at locus C permits attachment of a sequencing adaptor, while integration at locus D permits attachment of a tether to a bead or membrane E for example via oligonucleotide F, such as in the scheme shown in FIG. 14, F.

FIG. 28 shows an example of a scheme that permits the specific amplification of a region of interest. Two Cas-transposase cargos (A, B), each bearing a 3′-terminated oligonucleotide flap (C), are inserted at two loci in the configurations shown. Primers (D, E) complementary to these flaps are then used to amplify the region of interest by PCR or isothermal amplification yielding eventual product F. The cargos and primers may be identical or different. The primers may optionally introduce a barcode during the amplification step.

FIG. 29 shows an example of a scheme that permits the specific amplification of a region of interest and incorporates a unique molecular identifier (UMI) via a Cas-transposase-inserted cargo. The amplification scheme is as per FIG. 28, except with the addition of a UMI in the cargo sequence(s) (A, B). UMIs may additionally be added to the PCR product via the amplification primers.

FIG. 30 shows an example of a scheme that generates a linked template-complement sequencing read via a Cas-transposase-mediated insertion, with the aim of increasing single-molecule accuracy. In this example the cargo bears an oligonucleotide flap that bears a 3′ end in hairpin configuration E. A shows a possible product of integration. When extended by a strand-displacing polymerase (step B), for example Klenow exo-, the polymerase will duplicate the molecule (incorporated nucleotides shown as dotted lines) to its terminus, generating a template-complement linked, dA-tailed species C that can be ligated to a sequencing adaptor D. This molecule can then be sequenced using a nanopore. Block arrow shows read direction.

FIG. 31 shows an example of a scheme that generates a linked template-complement sequencing read via a Cas-transposase-mediated insertion, with the aim of increasing single-molecule accuracy. Example is as per FIG. 30, except with two Cas-transposase pairs inserting cargos at two sites, such that template-complement linked reads may be generated over a specific region of interest A. Block arrows show read direction.

FIG. 32 shows an example of how a Cas-transposase system may be used to generate a closed circular single-stranded DNA via a splint. Two cargos A and B bearing 5′ and 3′ oligonucleotide flaps G, H are inserted using a Cas-transposase system yielding species C. When species C is denatured in the presence of heat and alkali in the presence of splint D, the duplex strands separate and, under renaturing conditions, each flap partly anneals to splint D to yield a nicked, partly duplex species E. E may then be sealed to yield a single-stranded circular DNA F by a ligase, for example Taq DNA ligase. F may then be amplified by rolling-circle amplification using splint D or any other complementary oligonucleotide as a primer to generate concatemers of the sequence contained in product E.

FIG. 33 shows how two Cas-transposase pairs (A, B) may be used to insert Oxford Nanopore Technologies' 1D² adapters for sequencing a region of interest C. FIG. 34 shows how a Cas-transposase system may be used to introduce two hairpins at a break site. A, possible cargo design involving two hairpins. B shows how an oligonucleotide may be hybridized to both hairpins to physically link the hairpins together even after the introduction of a double-strand break. C shows the product of integration from cargo A. C may be prepared for sequencing by dA-taiing (D) and ligating sequencing adapters (E), yielding two molecules that each bear a hairpin F at the break site. Block arrows show read direction.

FIG. 35 shows how a pair of Cas-transposase complexes, each bearing a hairpin cargo, may be used to generate a circularly closed duplex molecule of a region of interest bearing a hairpin at both ends. A shows a possible cargo molecule with single hairpin species. Integration of cargo A into target B (bearing two integration sites on opposite strands) yields circularly closed product C with two hairpin ends. Background DNA (bearing one or no hairpins) may then be optionally digested using appropriate exonucleases (H) for example exonucleases III and VII. Product C may then be amplified using a primer D (bearing attachment site E) annealed to either or both hairpins, generating a molecule F that contains concatemers of the sequence of product C. Attachment of sequencing adapter G allows molecule F to be sequenced. Block arrow shows read direction.

FIG. 36 shows an alternative means to FIG. 34 of attaching a sequencing adapter to a target analyte using hairpins. In this case the hairpins contain a cleavable linker, for example deoxyuracils that can be cleaved by USER enzyme, or photocleavable linkers that can be cleaved using light. Cleavage of the hairpins yields a forked structure that can be used to attach sequencing adapters, for example by ligating sequencing adapters that carry an overhang complementary to the fork arms.

FIG. 37 shows how the insertion of a nuclease-resistant cargo may be used to enrich for a specific region of interest. Two Cas-transposase pairs, each carrying a series of oligonucleotide spacers, abasic sites, or phosphorothioate bonds (A) on one or both strands of the duplex, may be used to insert the nuclease-resistant region into target DNA, yielding B. Exonuclease digestion degrades non-target DNA, leaving a molecule C that can be prepared for nanopore sequencing by dA-tailing (step D) and ligating sequencing adapters (step E).

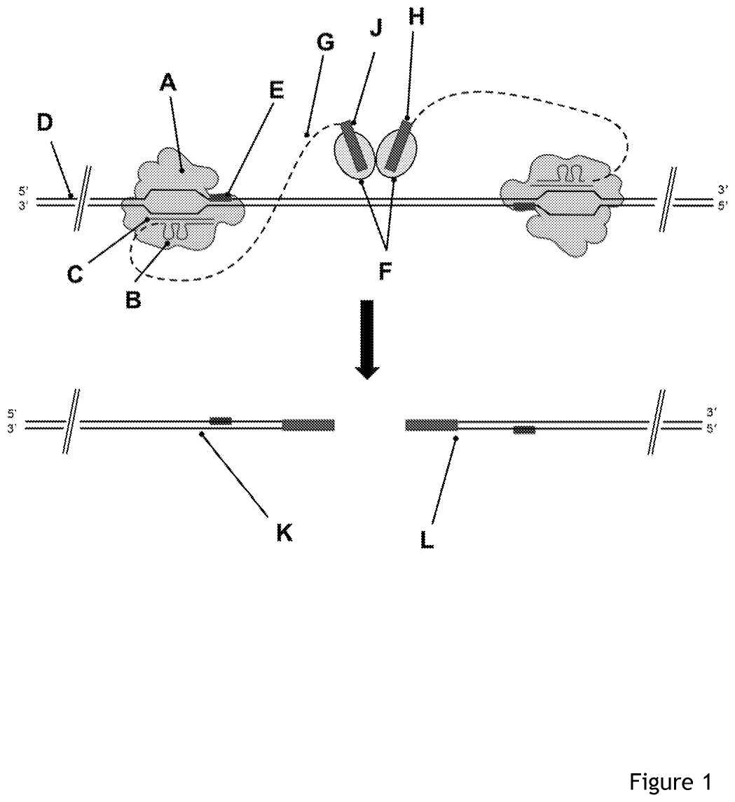

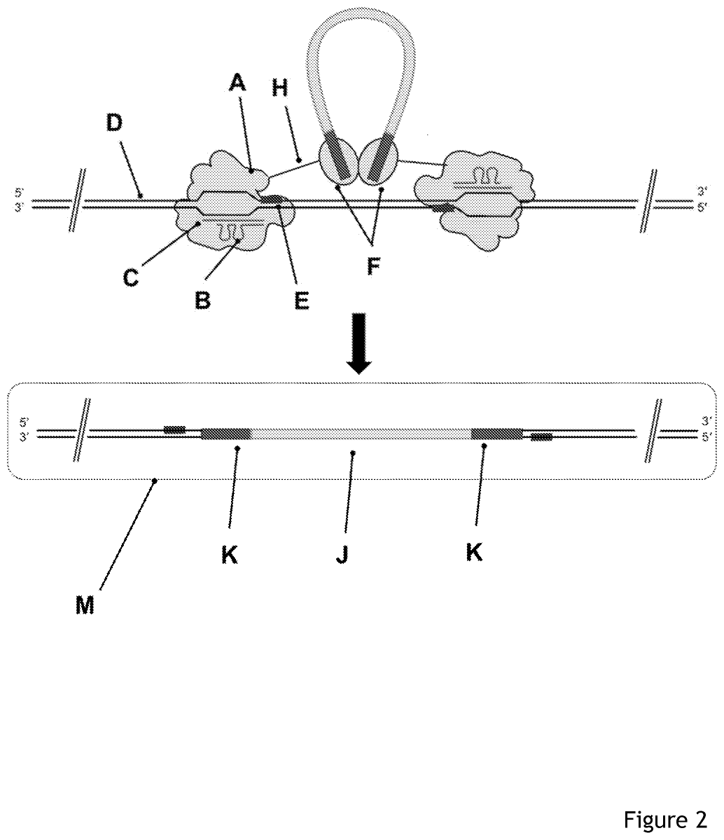

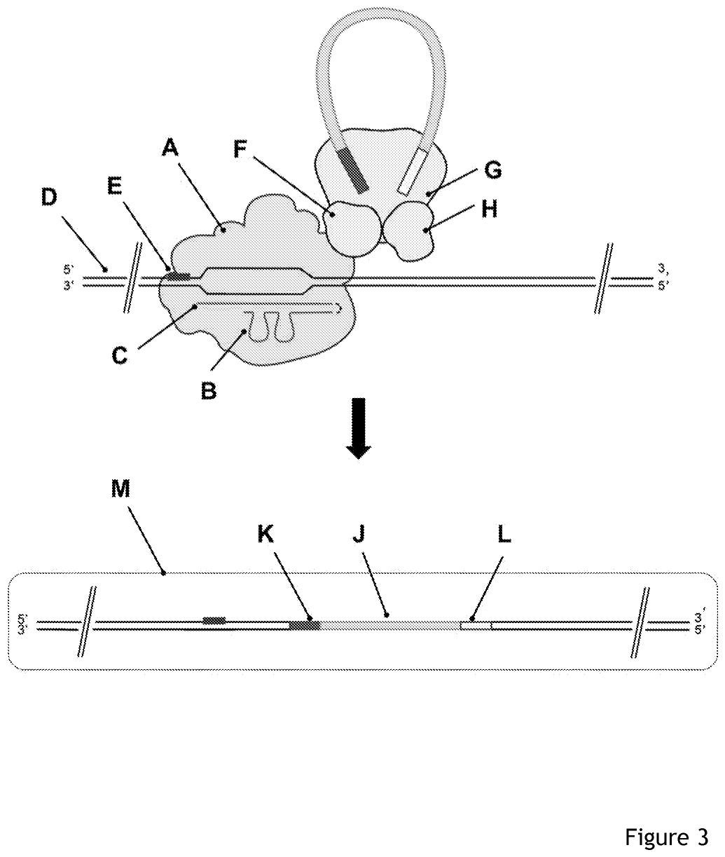

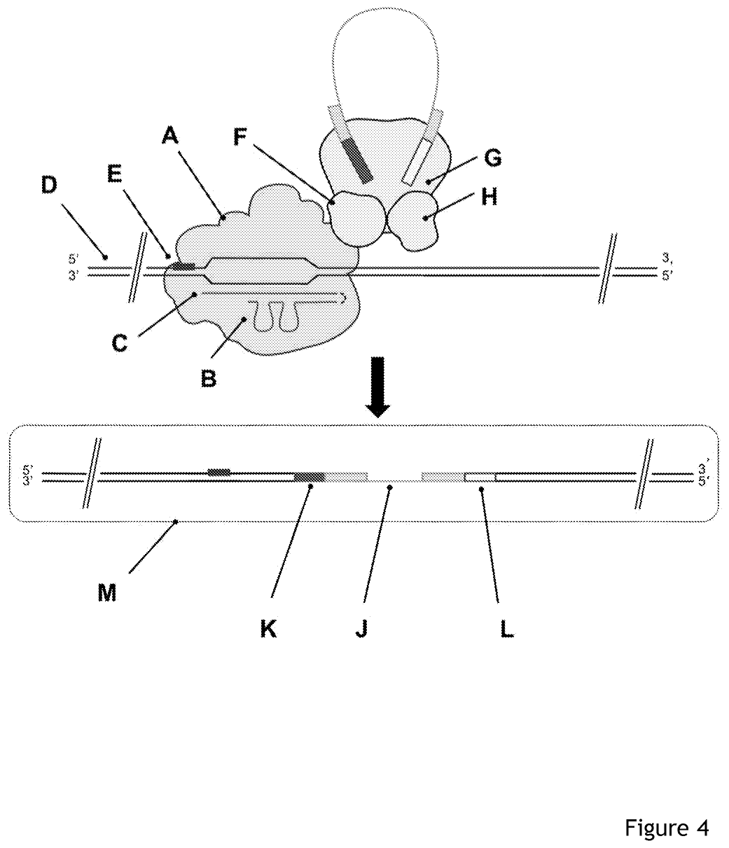

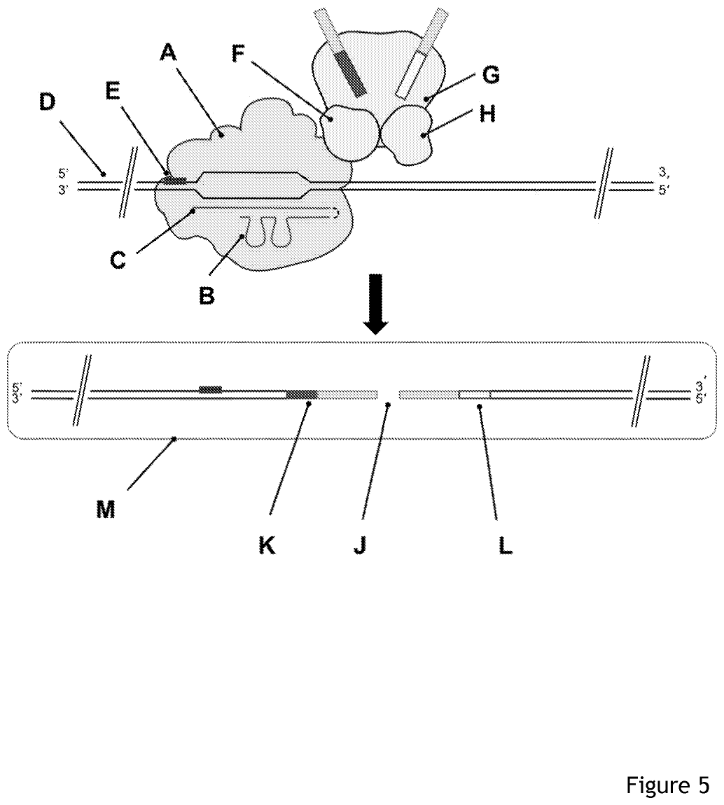

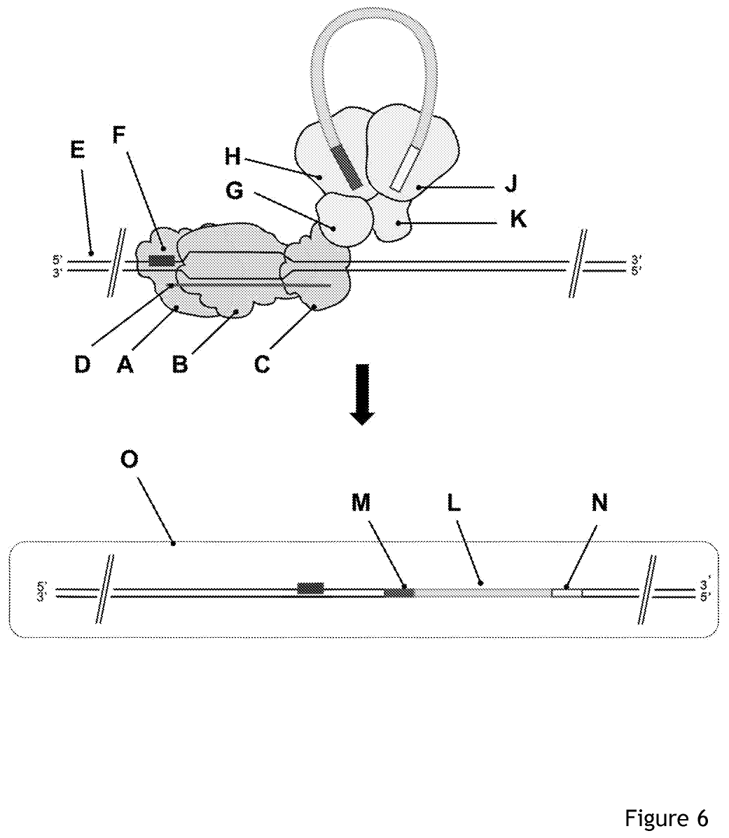

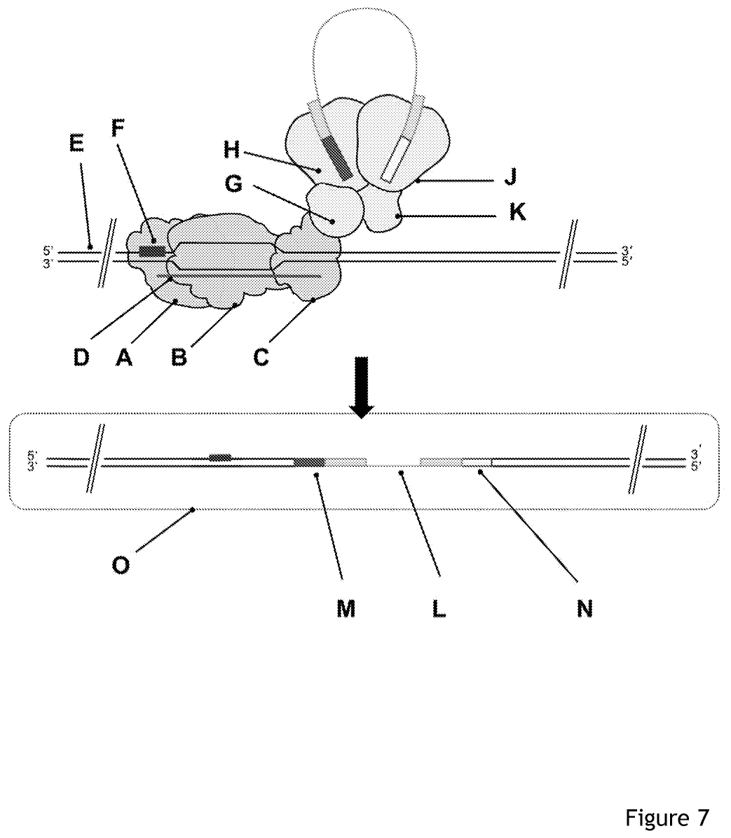

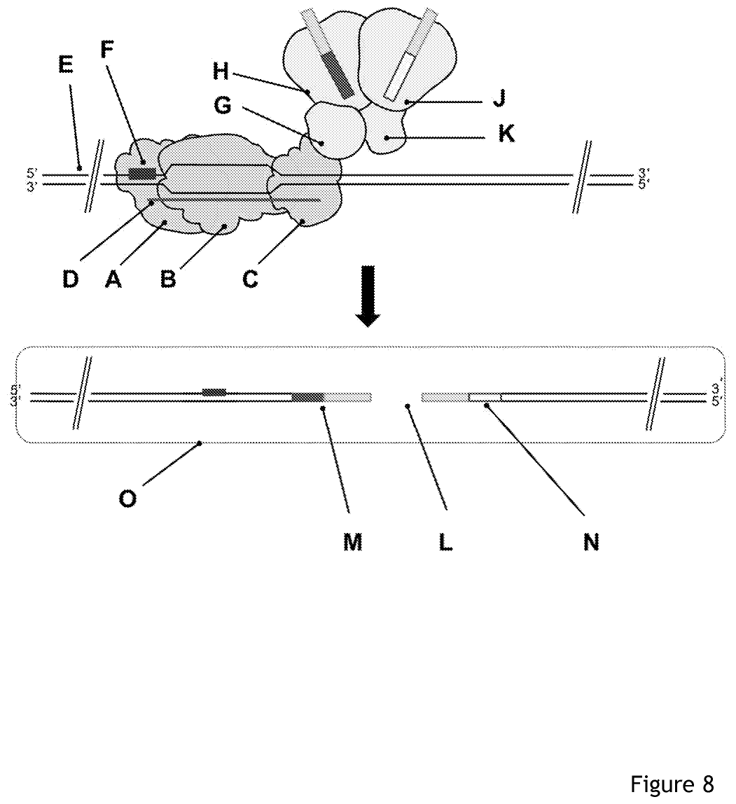

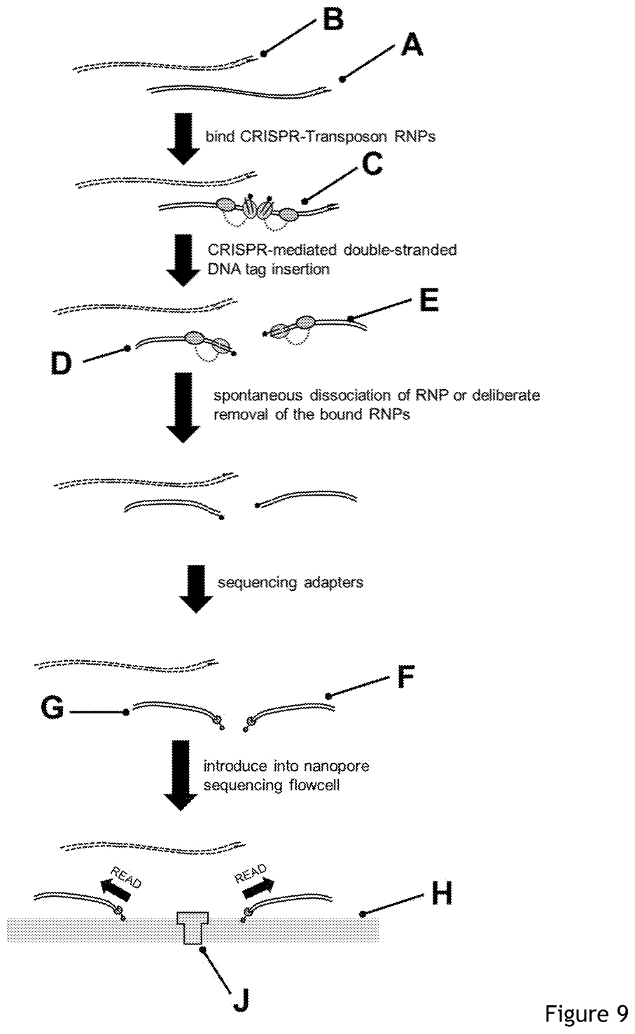

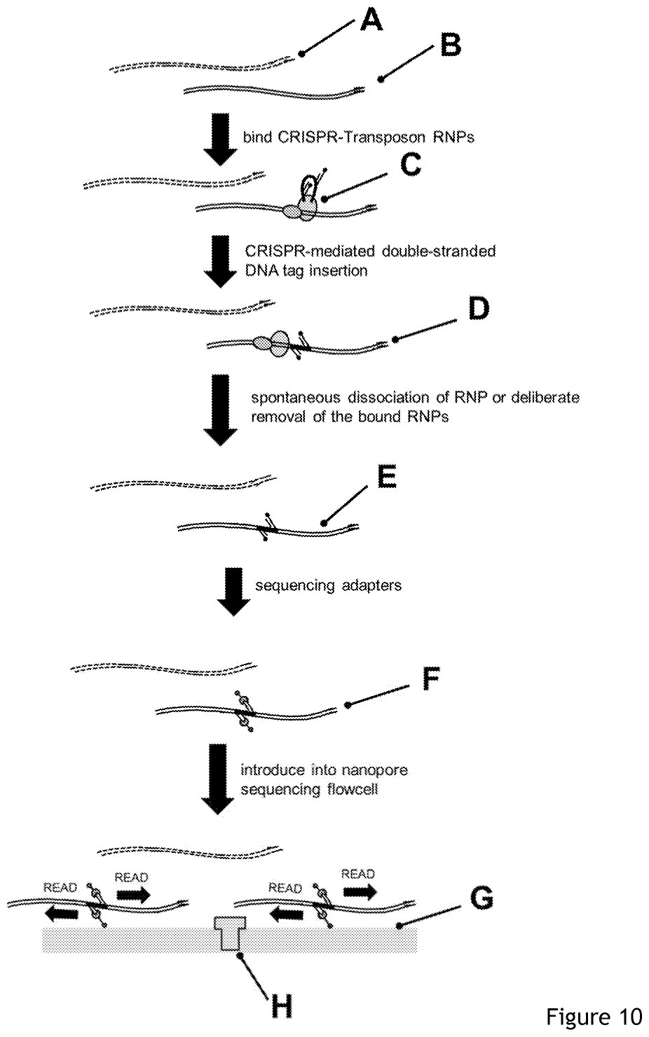

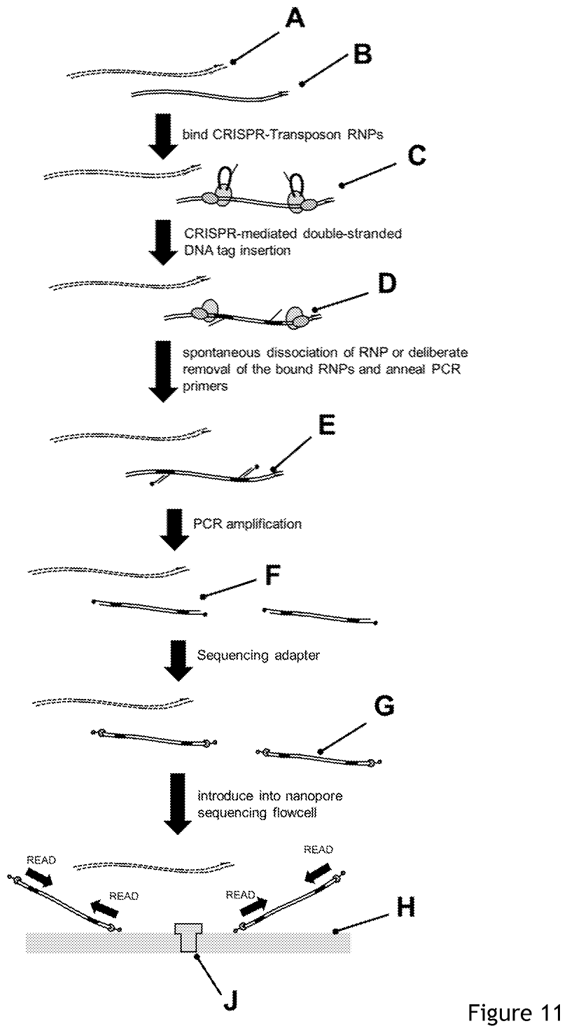

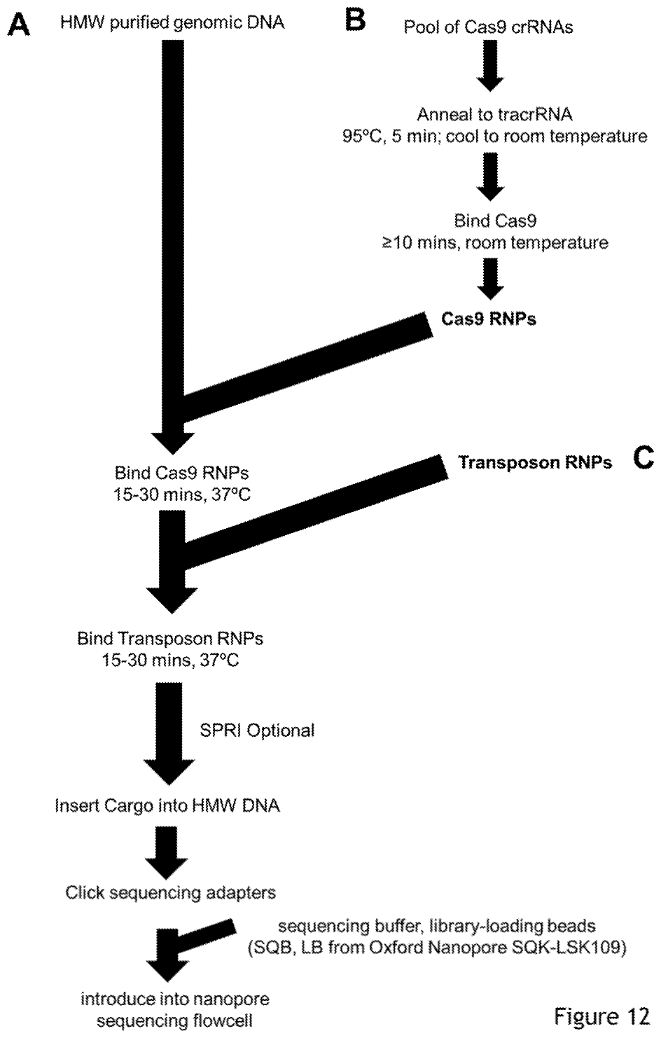

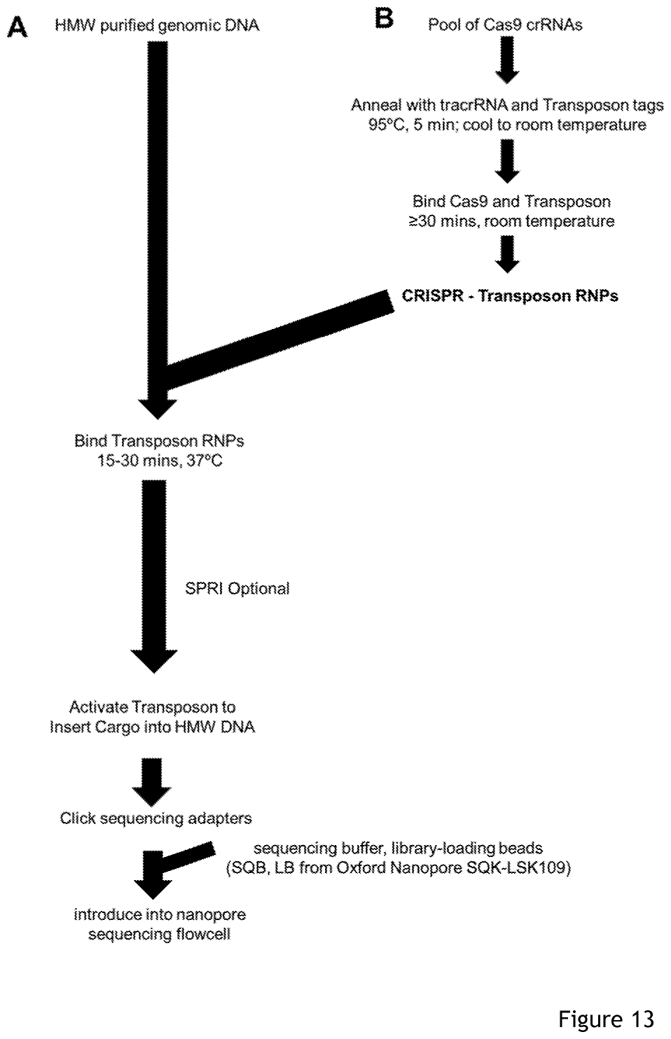

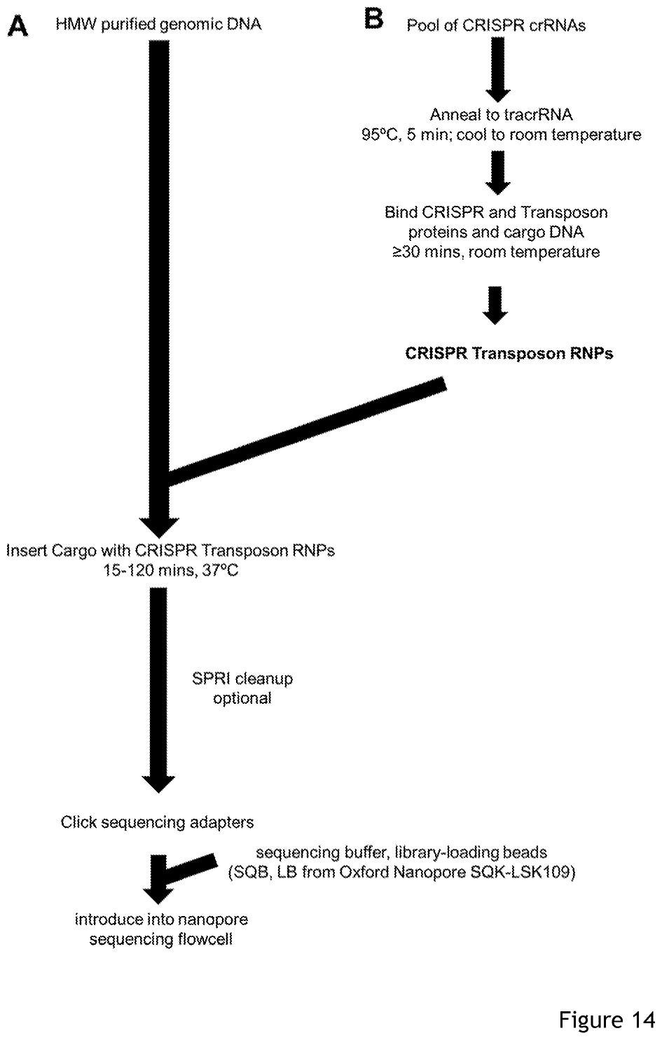

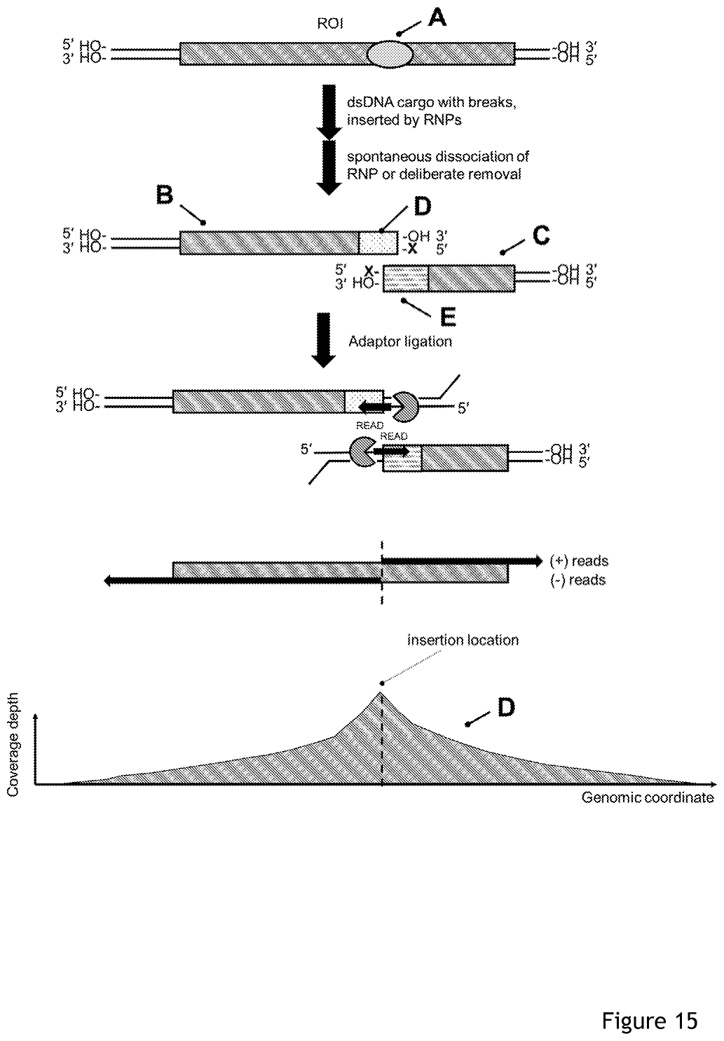

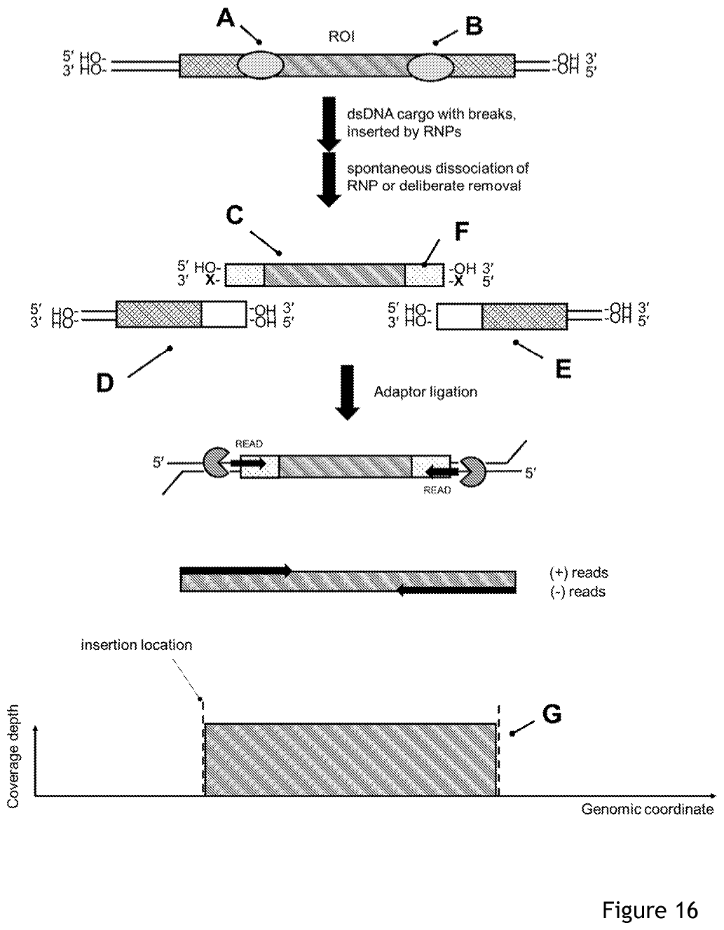

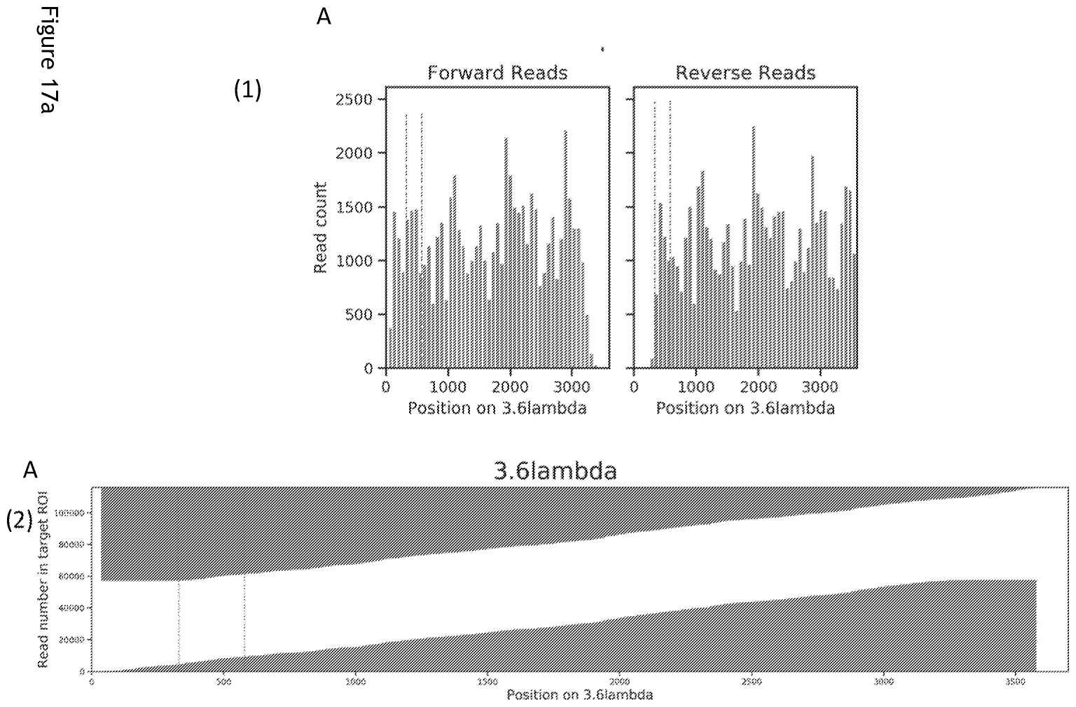

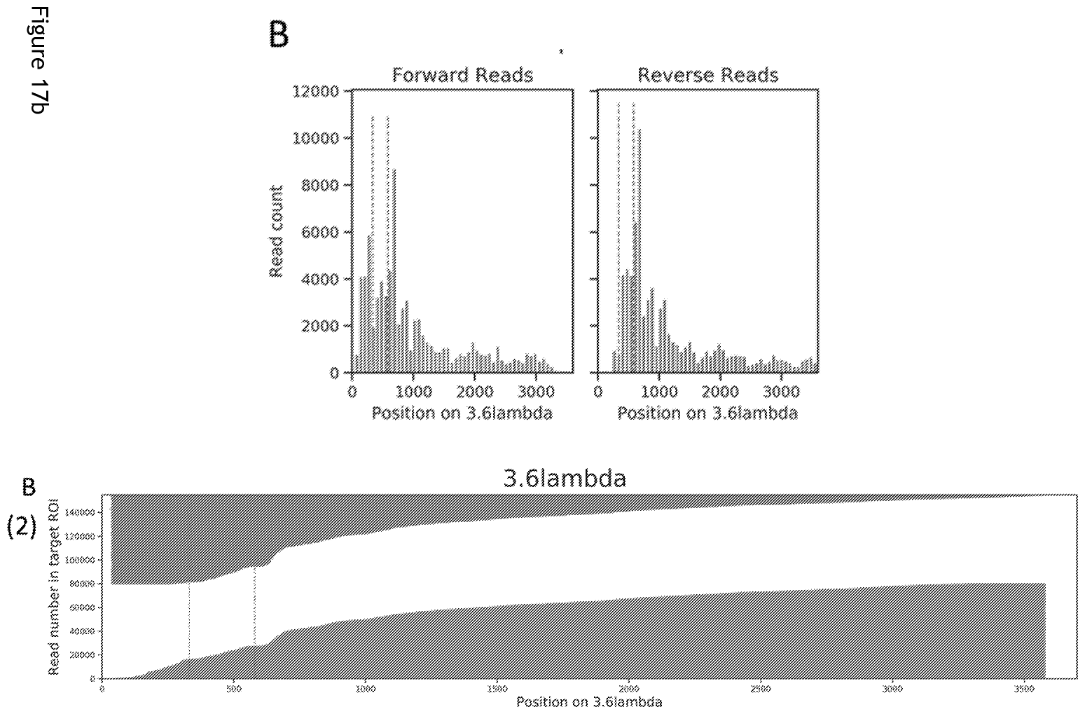

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polynucleotide” includes two or more polynucleotides, reference to a “molecule” refers to two or more and the like.

All publications, patents and patent applications cited herein, whether supra or infra, are hereby incorporated by reference in their entirety.

### Method of Preparing a Nucleic Acid Construct

The invention provides a method of preparing a nucleic acid construct for single molecule characterisation, comprising contacting a target polynucleotide with: a polynucleotide-guided effector protein (PGEP), a guide polynucleotide, a transposase and a transposable element comprising a modified polynucleotide, wherein the polynucleotide-guided effector protein directs said transposase to a region of interest within the target polynucleotide and the transposase inserts the transposable element into the polynucleotide, thereby producing a nucleic acid construct for single molecule characterisation.

In the method, the PGEP is used to direct a transposase to a region of interest within a target polynucleotide to be characterized. The transposase effects insertion of a transposable element to the region of interest. The transposable element comprises a modified polynucleotide. Accordingly, a modified polynucleotide is inserted to the region of interest. Modified polynucleotides are described in detail below but, in general, a modified polynucleotide may be an element that facilitates or improves single molecule characterization, such as a marker, a tether or an adaptor. Thus, the method modifies a target polynucleotide by inserting an advantageous modified polynucleotide in a region of interest, thereby preparing a nucleic acid construct for single molecule characterization.

The invention also provides a method of preparing a nucleic acid construct, comprising contacting a target polynucleotide with: a polynucleotide-guided effector protein, a guide polynucleotide, a transposase and a transposable element, wherein the polynucleotide-guided effector protein and transposase are genetically fused or connected via a linker moiety such that the transposase is directed to a region of interest within the target polynucleotide and inserts the transposable element into the target polynucleotide, thereby preparing a nucleic acid construct. The transposable element typically comprises a polynucleotide, and may comprise a modified polynucleotide.

In any of the methods described herein, the interaction between the PGEP and the transposase may be manipulated in order to tune the activity of the transposase. In this way, the accuracy of insertion can be improved, and off-target transposase activity minimized.

### Target Polynucleotide and Nucleic Acid Construct

As set out above, the method provides for the directed modification of a target polynucleotide. For example, the method may be used to prepare a nucleic acid construct for single molecule characterisation. The PGEP and transposase may be used to insert a transposable element comprising a modified polynucleotide into the target polynucleotide, thereby producing a nucleic acid construct for single molecule characterisation. The nucleic acid construct is therefore formed from the target polynucleotide and the transposable element.

The target polynucleotide and/or the nucleic acid construct may comprise a nucleic acid. The nucleic acid may comprise one or more naturally-occurring nucleic acids, such as deoxyribonucleic acid (DNA) and/or ribonucleic acid (RNA). The target polynucleotide and/or the nucleic acid construct may comprise single stranded DNA or single stranded RNA. The target polynucleotide and/or the nucleic acid construct may comprise double stranded DNA or double stranded RNA. The target polynucleotide and/or the nucleic acid construct may comprise a DNA/RNA duplex, e.g. one strand of RNA hybridized to one strand of DNA.

The nucleic acid may comprise one or more synthetic nucleic acids. Synthetic nucleic acids are known in the art. For example, the nucleic acid may comprises peptide nucleic acid (PNA), glycerol nucleic acid (GNA), threose nucleic acid (TNA), locked nucleic acid (LNA) and/or or other synthetic polymers with nucleotide side chains. If the polynucleotide is PNA, the PNA backbone may be composed of repeating N-(2-aminoethyl)-glycine units linked by peptide bonds. If the polynucleotide is GNA, the GNA backbone may be composed of repeating glycol units linked by phosphodiester bonds. If the polynucleotide is TNA, the TNA backbone may be composed of repeating threose sugars linked together by phosphodiester bonds. If the polynucleotide is LNA, the LNA backbone may be formed from ribonucleotides as discussed above having an extra bridge connecting the 2′ oxygen and 4′ carbon in the ribose moiety.

The target polynucleotide and/or the nucleic acid construct may be of any length. For example, the target polynucleotide and/or the nucleic acid construct may be at least about 10, at least about 50, at least about 70 at least about 100, at least about 150, at least about 200, at least about 250, at least about 300, at least about 400 or at least about 500 nucleotides in length. The target polynucleotide and/or the nucleic acid construct may be at least about 1,000, at least about 5,000, at least about 10,000, at least about 100,000 , at least about 500,000, at least about 1,000,000, or at least about 10,000,000 or more nucleotides in length. The target polynucleotide and/or the nucleic acid construct is preferably from about 30 to about 10,000, such as from about 50 to about 5,000, from about 100 to about 2,000 nucleotides, or about 500 to about 1,000 nucleotides in length. The target polynucleotide may itself be a fragment of a longer polynucleotide.

The target polynucleotide and/or the nucleic acid construct may be linear. The target polynucleotide and/or the nucleic acid construct may be circular. The target polynucleotide and/or the nucleic acid construct may an end-to-end RNA or DNA molecule.

The target polynucleotide may be comprised within a sample. The sample is typically one that is known to contain or is suspected of containing one or more polynucleotide molecules. The sample preferably comprises DNA and/or RNA. The sample may be a fluid sample.

The sample may be a biological sample, for example a sample from an animal, plant or virus. The sample may comprise one or more cells obtained from any organism or microorganism. The organism or microorganism is typically archaean, prokaryotic or eukaryotic. The organism or microorganism typically belongs to one of the five kingdoms: plantae, animalia, fungi, monera and protista.

The sample may be obtained from a human or a non-human animal. The human or animal may have, be suspected of having or be at risk of a disease. The non-human mammal may be a commercially farmed animal such as a horse, cow, sheep or pig. The non-human mammal may be a pet such as a cat or a dog. The sample may comprise a body fluid. The sample may be urine, lymph, saliva, mucus, seminal fluid, amniotic fluid, whole blood, plasma or serum.

The sample may be obtained from a plant. For example, the sample may be obtained from a commercial crop, such as a cereal, legume, fruit or vegetable. For example, the sample may be obtained from wheat, barley, oat, canola, maize, soya, rice, a banana, an apple, a tomato, a potato, a grape, tobacco, a bean, a lentil, a sugar cane, cocoa, cotton, a tea or coffee plant.

The sample may be a non-biological sample. Examples of non-biological samples may include water (such as drinking water, sea water or river water) and reagents for laboratory tests.

The sample may be processed prior to use in the method described herein. For example, the sample may be processed by centrifugation or by passage through a membrane that filters out unwanted molecules or cells, such as red blood cells. The sample may be stored prior to use in the method described herein. For example, the sample maybe stored below -70° C.

### Guide Polynucleotide

A polynucleotide-guided effector protein (PGEP) is a protein that is capable of binding to a target polynucleotide and a guide polynucleotide. In the method of preparing a nucleic acid construct, the PGEP is bound to a guide polynucleotide. The guide polynucleotide and PGEP typically form a complex, which then binds to the target polynucleotide at a site determined by the sequence of the guide polynucleotide. The guide polynucleotide is capable of hybridising to a complementary nucleotide sequence in the region of interest in the target polynucleotide. Hybridisation of the guide polynucleotide to the complementary nucleotide sequence in the regions of interest directs the PGEP to bind the desired region of interest. In turn, the PGEP is capable of directing the transposase to the region of interest.

The guide polynucleotide may comprise RNA and/or DNA. The guide polynucleotide preferably comprises RNA. The guide polynucleotide is preferably a guide RNA. Thus, the PGEP is preferably a RNA-guided effector protein. When the guide polynucleotide comprises RNA, the RNA may comprise crRNA (CRISPR RNA) and/or tracrRNA (transactivating CRISPR RNA). CRISPR RNAs and tracrRNAs are known in the art. The guide polynucleotide may comprise DNA. The guide polynucleotide may be a guide DNA. Thus, the PGEP may be a DNA-guided effector protein.

The guide polynucleotide comprises a sequence that is capable of hybridising to a region of interest within a target polynucleotide. The guide polynucleotide also comprises a sequence that is capable of binding to a PGEP. The sequence that is capable of hybridising to a region of interest within a target polynucleotide may be the same sequence as the sequence that is capable of binding to a PGEP, i.e. one sequence may bind both the region of interest in the target polynucleotide and the PGEP. The sequence that is capable of hybridising to a region of interest within a target polynucleotide may be a different sequence from the sequence that is capable of binding to a PGEP. As the guide polynucleotide is both capable of hybridising to a region of interest within the target nucleotide and binding to a PGEP, the guide polynucleotide is able to guide the PGEP to the region of interest. The guide polynucleotide may have any structure and sequence that enables these binding properties.

The sequence that is capable of hybridising to a region of interest within a target polynucleotide may be capable of hybridising to a sequence that is about 10 to about 40 nucleotides in length. For example, the sequence may be capable of hybridising to a sequence that is about 11 to about 39, about 12 to about 38, about 13 to about 37, about 14 to about 36, about 15 to about 35, about 16 to about 34, about 17 to about 33, about 18 to about 32, about 19 to about 32, about 20 to about 30, about 21 to about 29, about 22 to about 28, about 23 to about 27, about 24 to about 26, or about 25 nucleotides in length. The sequence may be capable of hybridising to a sequence that is about 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. The guide polynucleotide is typically complementary to one strand of a double stranded region of the target polynucleotide. The degree of complementarity is preferably exact.

The guide polynucleotide may comprise a crRNA and a tracrRNA. The crRNA may be a single stranded RNA. The crRNA may be capable of hybridising to a sequence in the region of interest within the target polynucleotide. The crRNA may be designed to target any region of interest. Methods for doing so are known in the art. The tracrRNA may comprise a stem-loop structure that is capable of binding to a PGEP. The tracrRNA may also hybridise to the 5′ or 3′ end of the crRNA. Thus, the crRNA typically binds to the target polynucleotide, and the tracrRNA typically binds to the PGEP. The crRNA and tracrRNA may be transcribed in vitro as a single polynucleotide, i.e. a single guide RNA (sgRNA). Thus, a sgRNA is a polynucleotide that may comprise a portion that is capable of hybridising to a region of interest in the target polynucleotide and another portion that is capable of binding to a PGEP.

### Polynucleotide Guided Effector Protein (PEGP)

As explained above, a polynucleotide-guided effector protein (PGEP) is a protein that is capable of binding to a target polynucleotide and a guide polynucleotide. In the method of preparing a nucleic acid construct, the PGEP is bound to a guide polynucleotide. The guide polynucleotide and PGEP typically form a complex, which then binds to the target polynucleotide at a site determined by the sequence of the guide polynucleotide. The guide polynucleotide is capable of hybridising to a complementary nucleotide sequence in the region of interest in the target polynucleotide. Hybridisation of the guide polynucleotide to the complementary nucleotide sequence in the regions of interest directs the PGEP to bind the desired region of interest. In turn, the PGEP is capable of directing the transposase to the region of interest.

The PGEP is capable of binding to a guide polynucleotide. Thus, the PGEP comprises a guide polynucleotide binding domain. A guide polynucleotide binding domain is a domain which is capable of binding to a guide polynucleotide. The PGEP (and, therefore, its guide polynucleotide binding domain) typically binds to a region of guide polynucleotide that is not capable of hybridising with the target polynucleotide. Guide polynucleotides are described in detail above. The guide polynucleotide may comprise DNA, in which case the PGEP is a DNA-guided effector protein. Exemplary DNA-guided effector proteins include proteins from the RecA, such as RecA, RadA and Rad51.

The guide polynucleotide may comprise RNA, in which case the PGEP is a RNA-guided effector protein. The RNA-guided effector protein is preferable a RNA-guided endonuclease, or an RNA-guided endonuclease whose nucleases activity is disabled.

The PGEP is capable of binding to a target polynucleotide. The PGEP may bind to a single stranded or double stranded region of the target polynucleotide. The PGEP may bind to the target polynucleotide upstream or downstream of the sequence to which the guide polynucleotide hybridises. The region of the target polynucleotide to which the PGEP binds is typically less than 100 nucleotides along the polynucleotide backbone from the site at which the guide polynucleotide hybridises to the target polynucleotide. The PGEP may bind to a protospacer adjacent motif (PAM) in the target polynucleotide. The PAM is preferably located less than 100 bases along the polynucleotide backbone from to the site at which the guide polynucleotide hybridises to the target polynucleotide. A PAM is a short sequence of less than 10 nucleotides. Typically, a PAM is 2 to 6 nucleotides in length. Suitable PAMs are known in the art. Exemplary PAMS include 5′-NGG-3′ (wherein N is any base), 5-NGTN-3, 5-GG-3, 5′-NGA-3′, 5′-YG-3′ (wherein Y is a pyrimidine), 5′TTN-3′ and 5′-YTN-3′. Different PGEPs bind to different PAMs. If a target polynucleotide does not comprise a PAM, the PGEP may bind elsewhere in target polynucleotide, as directed by the guide polynucleotide. A PAM is required for Cas binding.

The PGEP may comprise a target polynucleotide recognition domain. A target polynucleotide recognition domain is a domain that is capable of binding to a target polynucleotide. The target polynucleotide recognition domain may also be capable of binding to a guide polynucleotide.

The PGEP may comprise one or more nuclease domains. For example, the PGEP may comprise 2 or more, 3 or more, 4 or more or 5 or more nuclease domains. A nuclease domain is a domain which is capable of cutting or cleaving the target polynucleotide. When the target polynucleotide is single stranded, the nuclease domain is capable or cutting or cleaving the target polynucleotide at one or more points along the single strand. When the target polynucleotide is double stranded, the nuclease domain may be capable of cutting or cleaving at one or more points along one or both strands of the target polynucleotide. To effect cutting or cleavage of both strands of a double stranded target polynucleotide, the PGEP may contain one nuclease domain that is capable of cutting or cleaving both strands, or a first nuclease domain that is capable of cutting or cleaving one strand of the target polynucleotide and a second nuclease domains that is capable of cutting or cleaving the other (i.e. complementary strand) of the target polynucleotide.

The nuclease domain or domains comprised in the PGEP may be active or inactive. For example, the nuclease domain or domains may be inactivated by mutation. The ability of the PGEP to bind to the target polynucleotide via its target polynucleotide recognition domain may be unaffected by inactivity of the nuclease domain(s).

The nuclease activity of the PGEP may thus be disabled. Disablement may be, for example, by catalytic inactivation. For instance, one or more nuclease domains comprised in the PCT may be catalytically inactivated. As described above, the polynucleotide-guided endonuclease may comprise two nuclease domains, each capable of cutting or cleaving one strand of a double stranded target polynucleotide. In this case, one or both of the nuclease domains may be inactivated prior to use in the method, such that the PGEP may be capable of cutting or cleaving both strands, one strand or neither strand of a double stranded region of a target polynucleotide.

Nuclease activity may be disabled or inactivated by mutating a catalytic site comprised in the PGEP. The mutation may be a substitution, insertion or deletion mutation. For example, one or more (such as 2, 3, 4, 5, or 6 or more) amino acids may be substituted or inserted into, or deleted from, the catalytic site. The mutation is preferably an amino acid substitution, and more preferably a single amino acid substitution. The skilled person will be readily able to identify the catalytic sites of a PGEP and mutations capable of inactivating them. For example, where the PGEP is Cas9, a first catalytic site may be inactivated by a mutation at D10 and a second catalytic site may be inactivated by a mutation at H840.

The PGEP may comprise a single protein component. The PGEP may comprise an assembly of multiple protein components.

The PGEP may comprise a nuclease, such as a polynucleotide-guided endonuclease. The PGEP may comprise a Cas (CRISPR (clustered regularly interspaced short palindromic repeats) associated) protein. Any Cas protein known in the art may be used in the method. The polynucleotide-guided effector protein may comprise Cas, Csn2 (CRISPR associated protein Csn2), Cpf1 (CRISPR associated protein from Prevotella and Francisella 1), Csf1 (CRISPR associated protein Csf1), Cmr5 (CRISPR associated protein Cmr5), Csm2 (CRISPR associated protein Csm2), Csy1 (CRISPR associated protein Csy1), Cse1 (CRISPR E. coli associated protein) or C2c2. The Cas protein may be Cas3, Cas 4, Cas5, Cash, Cas7, Cas8 Cas8a, Cas8b, Cas8c, Cas9, Cas10 or Cas10d. Cas, Csn2, Cpf1, Csf1, Cmr5, Csm2, Csy1 or Cse1 is preferably used where the target polynucleotide comprises a double stranded DNA region. C2c2 is preferably used where the target polynucleotide comprises a double stranded RNA region. The PGEP may be Cas12k.

Preferably, the PGEP comprises Cas9. Cas9 has a bi-lobed, multi-domain protein structure comprising target recognition and nuclease lobes. The recognition lobe binds guide RNA and DNA. The nuclease lobe contains the HNH and RuvC nuclease domains which are positioned for cleavage of the complementary and non-complementary strands of the target DNA. The structure of Cas9 is detailed in Nishimasu, H., et al., (2014) Crystal Structure of Cas9 in Complex with Guide RNA and Target DNA. Cell 156, 935-949. The relevant PDB reference for Cas9 is 5F9R (Crystal structure of catalytically-active Streptococcus pyogenes CRISPR-Cas9 in complex with single-guided RNA and double-stranded DNA primed for target DNA cleavage).

The Cas9 may be an ‘enhanced specificity’ Cas9 that shows reduced off-target binding compared to wild-type Cas9. An example of such an ‘enhanced specificity’ Cas9 is S. pyogenes Cas9 D10A/H840A/K848A/K1003A/R1060A ONLP12296 is the amino acid sequence of S. pyogenes Cas9 D10A/H840A/K848A/K1003A/R1060A having a C-terminal Twin-Strep-tag with TEV-cleavable linker.

The PGEP may comprise Cash, Cas? and Cas8. Thus, the PGEP may comprise an assembly of Cas6-Cas7-Cas8 proteins. The PGEP may comprise Cas, Cpf1 and/or C2c2. Thus, the PGEP may comprise Cas. The PGEP may comprise Cpf1. The PGEP may comprise C2c2. The PGEP may comprise an assembly of Cas and Cpf1. The PGEP may comprise an assembly of Cas and C2c2. The PGEP may comprise an assembly of Cpf1 and C2c2. The PGEP may comprise an assembly of Cas, Cpf1 and C2c2. Any PGEP may additionally comprise Cas12k.

### Transposase

As described above, the PGEP functions to direct a transposase to the region of interest within the target polynucleotide. Once directed to the region of interest, the transposase functions to insert the transposable element, such as the transposable element comprising a modified polynucleotide, into the target polynucleotide.

Transposases are known in the art, and the skilled person would readily be able to identify a transposase for use in the method. Exemplary transposases families include DDE transposases, tyrosine (Y) transposases, serine (S) transposases, rolling-circle (RC) transposases, Y2 transposases and reverse transcriptases/endonucleases (RT/En). Particularly, exemplary DDE transposases include the maize Ac transposon, the Drosophila P element, Tn5, Tn7, Tn10, Mariner, IS10, IS50 or MuA. The transposase may comprise a single protein or protein monomer. The transposase may be a multimeric protein comprising a plurality of transposase proteins or transposase protein monomers. The multimeric transposase may be a homomultimer. For example, the transposase may comprise more than one MuA, more than one Tn5 or more than one Tn7 protein. The multimeric transposase may be a heteromultimer. For example, the transposase may comprise more than one different protein selected from MuA, Tn5 and Tn7 monomers, in any combination.

To effect its function, the transposase should be capable of interacting with a transposable element, such as a transposable element comprising a modified polynucleotide. The transposase may, for example, be capable of binding to the transposable element. The transposase may be capable of binding to more than one transposable element, wherein one or more of the transposable elements may comprise a modified polynucleotide. In this case, each transposable element may be the same as or different from the other transposable element(s) to which the transposase is capable of binding. The transposase may be capable of binding to the more than one transposable element simultaneously. The transposase may bind to a transposable element, such as a transposable element comprising a modified polynucleotide by binding to left-end (LE) and/or right-end (RE) motifs within the transposable element. LE and RE motifs are known in the art and may, for example, be inverted repeat sequences.

### Transposable Element

A transposase, when bound to a transposable element, such as a transposable element comprising a modified polynucleotide, may mediate insertion of the transposable element into a target polynucleotide. Transposable elements are described in detail below.

Transposable elements are known in the art (see, for example, Saariaho and Savilahti, Nucleic Acids Research, 2006; 34(10): 3139-3149 and Lee and Harshey, J. Mol. Biol., 2001; 314: 433-444), where they may alternatively be referred to as a transposase “cargo” or “payload” A a transposable element comprising a particular modified polynucleotide may be designed using methods known in the art. The skilled person could readily design and produce a transposable element comprising any polynucleotide, such as amodified polynucleotide, of interest.

The transposable element may comprise DNA and/or RNA. The transposable element may comprise a single stranded and/or a double stranded polynucleotide, or may be partially single- and partially double-stranded. The length in nucleotides of the transposable element may be vary depending on the nature of the modified polynucleotide comprised within the transposable element and/or on the particular purpose of the preparation of nucleic acid construct in the method of the invention. The transposable element may be more than 5, 10, 20, 30, 40, 50, 100, 500, 1000, 5000, 10000, 50000 or 100000 nucleotides in length. The preferred length of transposable element may also vary depending on the type of transposase being used. For example, the preferred length of substrate for MuA is between 5 and 5000 nucleotides in length, more preferably between 10 and 1000 nucleotides in length, and even more preferably between 20 and 200 nucleotides in length.

Transposable elements are generally capable of being joined to an exposed 5′ and/or 3′ end within a transposase-mediated nick in a strand of a single stranded or double stranded target polynucleotide, or to an exposed 5′ end and/or an exposed 3′ end within a transposase-mediated double-strand break of a double stranded target polynucleotide.

That is, a transposable element may be capable of being joined to an exposed 5′ end in a single strand, transposase-mediated nick. A transposable element may be capable of being joined to an exposed 3′ end in a single strand, transposase-mediated nick. A transposable element may be capable of being joined to an exposed 5′ end and an exposed 3′ end in a single strand, transposase-mediated nick. The transposable element may thus link an exposed 5′ end in a single strand, transposase-mediate nick to an exposed 3′ end in a single strand, transposase-mediate nick. In this way, a fractured or fragmented strand may be repaired by insertion of a transposable element in the nick.

A transposable element may be capable of being joined to one or both exposed 5′ ends in a transposase-mediated double strand break. A transposable element may be capable of being joined to one or both exposed 3′ ends in a transposase-mediated double strand break. A transposable element may be capable of being joined to one or both exposed 5′ ends and one or both exposed 3′ ends in a transposase-mediated double strand break. A transposable element may therefore be joined to both an exposed 5′ end and an exposed 3′ end in a transposase-mediated double strand break. Prior to contact with a transposase, the exposed 5′ end and the exposed 3′ end may have been attached, i.e. part of the same strand. In this way, a fractured or fragmented strand forming subject to a double strand break may be repaired by insertion of a transposable element. Joining may be by ligation.

When a transposable element is joined to an exposed 5′ or 3′ end of a transposase-mediated nick within a single strand of a double stranded target polynucleotide, an “overhang” or “flap” may result. In an overhang or flap, some or all of the transposable element that is joined to the exposed end of the nicked strand is not complementary to the sequence of the nicked or intact strand of the target polynucleotide upstream or downstream of the nick.

The transposable element typically comprises left-end (LE) and/or right-end (RE) motifs. LE and RE motifs are known in the art and may, for example, be inverted repeat sequences. A polynucleotide of interest, such as a modified polynucleotide may, for example, be inserted between the LE and RE motifs in the transposable element.

### Modified Polynucleotide

The transposable element may comprise one or more polynucleotide, such as one ore moremodified polynucleotide. A modified polynucleotide may be any element that assists in single molecule characterization of the target polynucleotide. For example, a modified polynucleotide may be an element that, following insertion into the target polynucleotide, may be manipulated, modified and/or detected. For example, the modified polynucleotide may comprise one or more click reactive group, one or more fluorophore, one or more conjugation agent, one or more pull down group, one or more tethering moiety, one or more marker, one or more modified base, one or more abasic residue and/or one or more spacer. For example, 2, 3, 4, 5, 6, 7, 8, 9 or 10 such modifications may be included in the modified polynucleotide.

The modified polynucleotide may be a marker. The marker may be any suitable marker that enables the skilled person to identify where or whether the one or more transposable elements have been inserted into the region of interest in the target polynucleotide. Exemplary markers include one or more biotin molecules; one or more modified bases; one or more abasic residues; one or more base-base conjugates; or one or more protein-base conjugates.

Preferably, the marker is detectable by one or more sequencing technologies known in the art, such as by nanopore-based sequencing. The marker may be optically detectable. For example, the marker may fluoresce under excitation by the appropriate wavelength of light. For example, the marker may comprise one or more fluorescent bases, for example Cy3 or Cy5. Any optical and/or fluorescent marker deemed suitable by the skilled person may be used. Other detectable markers include non-canonical bases, abasics and spacers. Any abasic and/or spacer deemed suitable by the skilled person may be used. Particularly, exemplary spacers may include C3, PC Spacer, Hexanediol, Spacer 9, Spacer 18 or 1′,2′-Dideoxyribose (dSpacer).

The modified polynucleotide may comprise one or more modified bases. The modified base can be any suitable modified base. The modified base may be, for example, a nucleotide labelled with biotin (i.e. a biotinylated nucleotide), or a nucleotide labelled with digoxigenin (i.e. a digoxigenin-labelled nucleotide). Modified bases such as biotin-labelled or digoxigenin-labelled bases may enable coupling of the nucleic acid constructs to a solid surface, for example a surface coated with streptavidin or anti-digoxigenin respectively.

The modified polynucleotide may comprise a pull-down group. The pull-down group may be any suitable pull-down group that enables the skilled person to purify or isolate the nucleic acid construct or immobilize the nucleic acid construct by attaching the construct to another substance. The other substance may, for example, be a nucleic acid construct, a nucleic acid molecule, a polypeptide, a protein, a membrane or a solid phase surface. Exemplary pull-down groups include one or more polypeptides, and one or more hydrophobic anchors.

The pull-down group may, for example, comprise one or more modified nucleotide bases. The modified base may be a nucleotide labelled with biotin (i.e. a biotinylated nucleotide), or a nucleotides labelled with digoxigenin (i.e. a digoxigenin-labelled nucleotide). Modified bases within the pull-down group such as biotin- or digoxigenin-labelled bases may enable tethering of the nucleic acid constructs to a solid surface, for example, a surface coated with streptavidin or anti-digoxigenin. Any suitable tether that enables coupling to a solid surface may be used. Solid surfaces that could be coupled to nucleic acid constructs prepared by the methods of the invention described herein may, for example, include nanogold, polystyrene bead and Qdot.

The pull-down group may, for example, comprise a tethering moiety wherein the tethering moiety comprises a hydrophobic anchor, optionally wherein the hydrophobic anchor comprises a hydrophobic nucleotide base. The tethering moiety may be a lipid, fatty acid, sterol, carbon nanotube, protein or amino acid, cholesterol, palmitate or octyl tocopherol.

The modified polynucleotide may be an adaptor. The adaptor may permit further manipulation of the nucleic acid construct or may permit direct sequencing of the nucleic acid construct. The adaptor may be any adaptor deemed suitable by the skilled person.

Adaptors are known in the art. The adaptor may, for example, comprise a nucleotide sequence enabling protein binding, a sequencing adaptor, a PCR adaptor, a hairpin adaptor, an adaptor that would enable circularization of a target polynucleotide and/or rolling circle amplification, a unique molecular identifier (UMI), an oligonucleotide splint, a click chemistry moiety, a exonuclease-resistant bases and/or phosphorothioate bonds. The adaptor may concurrently be bound by a desired protein, for example, a motor enzyme.

The adaptor may comprise RNA and/or DNA sequences that can be recognized and bound by a DNA and/or RNA binding protein. The adaptor may be bound by a DNA and/or RNA binding protein prior to, or following, insertion of said adaptor into the target polynucleotide. The RNA and/or DNA binding protein bound to the adaptor may be capable of binding double- and/or single-stranded polynucleotides. For example, in the method described herein, the adaptor may be bound by a motor enzyme such as a helicase or a translocase.

The adaptor may be a sequence motif that may be specifically recognized by a particular DNA and/or RNA binding protein. The adaptor may be an RNA/DNA hybrid sequence that may be specifically recognized and bound by a DNA and/or RNA binding protein. For instance, sequence motifs may be recognized by DNA-binding proteins characterised by their structural domains e.g. helix-turn-helix, zinc finger, leucine zipper, winged helix, winged helix-turn-helix, helix-loop-helix, HMG-box, Wor3 and OB-fold. The adaptor may be a lac( )or tet0 array capable of being bound by lac or tet repressor proteins. The adaptor may be an RNA-DNA hybrid sequence capable of being bound by an antibody. The RNA-DNA hybrid marker may be bound by a S9.6 antibody.

The adaptor may comprise a nucleotide sequence suitable for oligonucleotide hybridisation. Particularly, oligonucleotides may comprise complementary bases for hybridizing to the adaptor, thus enabling priming of the extension (linear amplification) of a complementary polynucleotide sequence or polymerase chain reaction. For example, in any of the methods described herein, two PGEP-transposase pairs may target distinct regions of interest on opposite strands of a target double-stranded polynucleotide. Each PGEP-transposase may then insert an adaptor comprising an oligonucleotide overhang further comprising a sequence providing a hybridisation site for a primer, thus enabling antiparallel amplification of a region between the two regions targeted by each PGEP-transposase.

The adaptor may comprise a nucleotide sequence that may act as a unique molecular identifier (UMI). The UMI may be detectable by any sequencing method deemed suitable by the skilled person. Particularly, the UMI may be used to detect and quantify the nucleic acid construct. Nucleic acid construct sequencing reads may be clustered by the presence of a UMI, thus enabling the improvement of single molecule sequencing accuracy.

The adaptor may comprise a hairpin moiety. Hairpin adaptors are known in the art. Particularly, the adaptor may comprise a hairpin that has inserted into the target polynucleotide via the 5′ end of the hairpin. The hairpin may have a free 3′ end, wherein a templated extension of the hairpin may be mediated by the polymerase to generate a complementary 2D strand. Other exemplary applications of adaptors comprising hairpin moieties in the context of the invention described herein involve the insertion of hairpin adaptors at both of the exposed ends of a PGEP-transposase-mediated double strand break in a target polynucleotide. The hairpins may optionally be linked by a cleavable linker moiety. DNA motors and adaptor moieties may then be attached to the non-hairpin ends of the target polynucleotide for direct application to a nanopore-based sequencing protocol for 2D sequencing. In another application, two PGEP-transposase pairs may target distinct regions of interest within a target double-stranded polynucleotide. Each PGEP-transposase may mediate a double-strand break and insert hairpin adaptors in order to generate a circularly closed molecule spanning the two regions of interest to form a nucleic acid construct amenable to rolling circle amplification. A further example may involve the use of an exonuclease-resistant hairpin thus enabling the preservation of the nucleic acid constructs prepared by the methods of the invention whilst other background polynucleotides may be digested by exonucleases. A further example may involve the use of hairpins comprising a uracil or other groups e.g. photocleavable spacers, that permit the cleavage of the hairpin, optionally after exonuclease digestion of background polynucleotides, and subsequent ligation of sequencing adaptors and optionally DNA motors.

The adaptor may be any adaptor amenable to form a covalent bond to other molecules, for example via click chemistry. The adaptor comprised in the modified polynucleotide may comprise a group that allows copper-free click chemistry. An exemplary group applicable to the adaptors of the present method is a 5′DBCO group. For example, in any of the methods described herein, two PGEP-transposase pairs may target distinct regions of interest on opposite strands of a target double-stranded polynucleotide. Each PGEP-transposase may then insert an adaptor comprising an oligonucleotide overhang further comprising a bound DNA motor and/or a sequence providing a click chemistry moiety for covalent bonding of a nanopore-based sequencing adaptor, thereby allowing direct application to a nanopore-based sequencing protocol.

A pair of adaptors inserted into the same strand of a double-stranded oligonucleotide may act as “splint” in the method described herein. A splint in the context of the present disclosure involves PGEP-transposase contacting two distinct regions of any distance along the same strand of a double-stranded target polynucleotide and introducing a nick in the same strand in each of the two regions of interest. An adaptor is then attached to the nicked strand at each region of interest. The adaptor attached to the nick in the 5′ region of interest is attached to the nicked strand via the adaptor's 3′ end, thus resulting in a 5′ overhang, wherein the 5′ end of the adaptor is an exposed phosphate group. The adaptor attached to the nick in the 3′ region of interest is ligated to the nicked strand via the adaptor's 5′ end, thus resulting in a 3′ overhang, wherein the 3′ end of the adaptor is an exposed hydroxyl group. Upon denaturation of the target polynucleotide, the portion of comprising the ligated substrates may be ligated to a further polynucleotide molecule to form a circular polynucleotide molecule. The circular polynucleotide may be amplified by rolling circular amplification.

Any modified polynucleotide may be capable of further modification.

Relationship between the PGEP and the Transposase

The method involves contacting a target polynucleotide with a PGEP, a transposase and a transposable element. Such contacting allows the PGEP to direct the transposase to a region of interest within the target polynucleotide, to effect insertion of a transposable element, optionally comprising a modified polynucleotide into the region of interest.

The PGEP may bind to the transposase in order to direct it to the region of interest. In other words, binding between the PGEP and the transposase may determine the locus at which the transposase contacts the target polynucleotide. The nature of such binding may determine the distance between the point at which the PGEP contacts the target polynucleotide and the point at which the transposase contacts the target polynucleotide.

Binding between the PGEP and the transposase may be direct or indirect. For example, the binding between the PGEP and the transposase may be mediated by one or more protein-protein interactions. The PGEP and the transposase may be genetically fused. The binding between the PGEP and the transposase may be mediated by one or more linker moieties.

When binding between the PGEP and the transposase is mediated by one or more linker moieties, the linker moiety may be linker polynucleotide that binds to the PGEP and to the transposase. The linker polynucleotide is not the target polynucleotide. The linker polynucleotide may comprise any kind of polynucleotide, such as DNA and/or RNA. The linker polynucleotide may be of any length. The linker polynucleotide may comprise tracrRNA, a CRISPR RNA or single guide RNA. An individual linker polynucleotide may bind the PGEP and the transposase. Alternatively one or more linker polynucleotides may bind the PGEP and hybridise with one or more further linker moieties that are bound to the transposase.

The linker moiety sequence length may determine the length of the sequence between (i) a protospacer adjacent motif (PAM) immediately upstream of the sequence within the target polynucleotide contacted by the polynucleotide-guided effector protein and (ii) the site in which the transposase inserts the transposable elementinto the region of interest. In other words, a particular linker length may enable the skilled person to control the distance between a PAM sequence immediately upstream of the sequence within the target polynucleotide contacted by the PGEP and the site in which the transposase inserts the transposable element into the region of interest. Increasing the length of the linker moiety would increase the distance between such a PAM sequence and the site of insertion. Thus, regions of interest within a target polynucleotide are not limited by the proximity of a site (e.g. a PAM) capable of being efficiently hybridized by a guide polynucleotide. Ideally, a sequence in the region of interest that is targeted by a guide polynucleotide will have minimal sequence identity with other parts of the target polynucleotide in order to minimize off-target guide sequence hybridization and PGEP binding. However, in some cases, a PAM may be required to be immediately upstream or downstream of the region of interest in order to achieve effective insertion of the transposable element. By varying the length of the linker that binds the PGEP to the transposase, the requirement for a nearby PAM may be offset, allowing greater freedom in the design of the guide polynucleotide.

The PGEP and the transposase may be in complex. Thus, the target polynucleotide may be contacted with a complex comprising the PGEP and the transposase. The complexed PGEP and the transposase may be genetically fused. The complexed PGEP and the transposase may be bound to each other. Binding is described above. The complex may be formed by binding the PGEP to the transposase and removing any transposase protein not bound the PGEP. Unbound transposase may be removed by isolating the PGEP or the transposase by an affinity purification technique.

As set out above, the PGEP and/or the transposase may comprise an assembly of multiple protein components. For example, the PGEP may comprise an assembly of one or more PGEPs, such as 2 or more, 3 or more, 4 or more, 5 or more, 6 or more, 7 or more, 8 or more, 9 or more, or 10 or more PGEPs. Each PGEP may be the same or different. The transposase may comprise an assembly of one or more transposase proteins, such as 2 or more, 3 or more, 4 or more, 5 or more, 6 or more, 7 or more, 8 or more, 9 or more, or 10 or more transposase proteins. Each transposase protein may be the same or different. By varying the number of protein components in the PGEP and/or the transposase, the stoichiometry of the PGEP and transposase may. The number of transposable element molecules bound by each transposase may also be varied. For example, the transposase may bind to one or more transposable element, such as 2 or more, 3 or more, 4 or more, 5 or more, 6 or more, 7 or more, 8 or more, 9 or more, or 10 or more transposable elements. Each transposable element may be the same or different.

Exemplary stoichiometric combinations of PGEP-transposase-transposable element include:
one PGEP and a pair of transposases, wherein the transposase pair together carries a single substrate e.g. a polynucleotide with a bound DNA motor;
one PGEP and a pair of transposases, wherein each transposase in the transposase pair carries a pair of substrates e.g. a pair of polynucleotides each with a bound DNA motor;
two PGEPs and two pairs of transposases, wherein each pair carries a single polynucleotide substrate e.g. one polynucleotide each with a bound DNA motor per pair of transposases;
two PGEPs and two pairs of transposases, wherein each transposase in the transposase pair carries a pair of substrates e.g. a pair of polynucleotides per pair of transposase each with a bound DNA motor.

### Contacting the Target Polynucleotide

The target polynucleotide is contacted with a PGEP, a transposase and a transposable element, such that the PGEP directs a transposase to a region of interest within the target polynucleotide to effect insertion of a modified polynucleotide comprised in the transposable element into the region of interest.

The conditions of the contacting step may be controlled so as to control the activity of the transposase. It may be advantageous to control the activity of the transposase to:
i) prevent the transposase contacting the target polynucleotide outside of any region of interest; and/or
ii) to prevent transposase-mediated insertion of one or more transposable element into the target polynucleotide by the transposase outside of any given region of interest.

Transposase activity may be controlled in any way. Exemplary, and non-limiting, methods of controlling transposase activity comprise:
i. sequential contacting of the target polynucleotide with the PGEP and transposase;
ii. light-activated control;
iii. inhibition of transposase enzymatic activity; and/or
iv. kinetic control.

i. Sequential Contacting of the Target Polynucleotide with the PGEP and Transposase

The target polynucleotide may be contacted with the PGEP, the transposase and the transposable element simultaneously. The target polynucleotide may be contacted with the PGEP, the transposase and/or the transposable element at different times. For example, the target polynucleotide may be first contacted with the polynucleotide-guided effector protein and then secondly contacted with the transposase. In other words, the target polynucleotide may be sequentially contacted with the PGEP and the transposase. For example, the PGEP may be added to a reaction mixture comprising the target polynucleotide. The transposase and the transposable element may then be added to the reaction mixture. The transposase and the transposable element may be added together or separately. During sequential contacting of the target polynucleotide with the PGEP and transposase, any transposase that is not bound to the PGEP is removed. Sequential contacting of the target polynucleotide with the PGEP and the transposase is advantageous, because the transposase is able to bind to PGEP that has already bound to the region of interest in the target polynucleotide. This minimizes the likelihood that the transposase will bind, and/or insert a transposable element at, a region outside of the region of interest.

During sequential contacting of the target polynucleotide with the PGEP and transposase, different reaction conditions may be used for contact with the PGEP and contact with the transposase. The target polynucleotide may be contacted with the PGEP under a first set of conditions, and with the transposase under a second set of conditions. The first set of conditions may be unfavourable to transposase activity. The second set of conditions may be favourable to transposase activity. Furthermore, any transposase that is not bound to the PGEP may be removed prior to the conditions being changed so they are favourable to transposase activity.

ii. Light Activated Control

Transposase activity in the context of the present invention may be controlled by light. Particularly, wherein the PGEP binds the transposase via one or more linker moieties, the one or more linker moieties may be subject to light-activated control, thus enabling the control of transposase activity by restricting its ability to contact the target polynucleotide. Light-activated control may involve photoswitching, thus meaning that a moiety amenable to light-activated control may reversibly activated and deactivated by light.

Examples of moieties susceptible to light-activated control include reversibly fluorescent proteins such as Dronpa, in addition to photoswitchable dyes such as azobenzene. Other moieties susceptible to light-activated control are known in the art.

The linker moiety that binds to the PGEP and to the transposase may undergo an ordered to disordered transition upon exposure to particular sources and/or wavelengths of light. The disordered linker moiety may cause the transposase to be less proximal to the target polynucleotide whilst the transposase remains bound to the linker moiety, or the disordered linker moiety may prevent binding of the transposase to the linker moiety at all. Thus, there may be light conditions that are favourable for evoking an ordered linker moiety, whilst there may be unfavourable conditions for evoking an ordered linker moiety. These conditions may be modulated in order to exert control over the activity of the transposase. A disordered linker moiety may prevent the transposase contacting the target polynucleotide outside of any region of interest; and/or to prevent transposase-mediated insertion of one or more transposable elements into the target polynucleotide by the transposase outside of any given region of interest.

The transposase may be caged by a light-activated polymeric network. Polymeric networks are known in the art. The monomers that form part of the polymeric network may be light-activated. Particularly, the monomers may be individually susceptible to light-activated control. Thus, stimulation of polymeric network may induce conformational changes in the network that leads to the release, and subsequent activation, of the caged transposase. The caging of the transposase may prevent binding of the transposase to the PGEP, thus preventing the transposase contacting the target polynucleotide outside of any region of interest; and/or to prevent transposase-mediated insertion of one or more transposable elements into the target polynucleotide by the transposase outside of any given region of interest.

iii. Inhibition of Transposase Enzymatic Activity

Transposase enzymatic activity may be controlled by the use of reaction conditions that are unfavourable or favourable to transposase activity. Conditions may be modulated activate or improve, or deactivate or decrease, the enzymatic activity of the transposase.

Accordingly, the target polynucleotide may be contacted with the polynucleotide-guided effector protein, the transposase and transposable element under conditions unfavourable to transposase activity.

Conditions that are unfavourable to transposase activity may reduce the capacity of the transposase to bind to the PGEP and/or the target polynucleotide, or to effect insertion of a transposable element. Conditions that are unfavourable to transposase activity are known in the art, and include low salt concentrations and high salt concentrations, and the presence of metal ion chelating agents.

Particularly, conditions wherein the salt concentration is no more than 50 mM or at least 250 mM are unfavourable to transposase activity. Thus, the conditions unfavourable to transposase activity may comprise a salt concentration that is no more than 50 mM or at least 250 mM. For example, the conditions unfavourable to transposase activity may comprise a salt concentration of about 1 mM to about 50 mM, such as 5 mM to 45 mM, 10 mM to 40 mM, or 20 mM to 35 mM. The salt concentration may, for example, be about 1mM, 2mM, 3mM 4mM, 5mM, 10mM, 20mM, 25mM, 30mM, 35mM, 40 mM, or 50 mM. The conditions unfavourable to transposase activity may comprise a salt concentration of 250 mM or more, 300 mM or more, 500 mM or more, or 1 M or more. Most preferably, the salt concentration is about 100 mM.

The conditions unfavourable to transposase activity comprise the presence of a metal ion chelating agent. Metal chelating agents are known in the art. An exemplary metal ion chelating agent is EDTA (ethylenediaminetetraacetic acid).

When the target polynucleotide is contacted with the polynucleotide-guided effector protein, the transposase and transposable element under conditions unfavourable to transposase activity, any transposase that is not bound to the polynucleotide-guided effector protein may be removed prior to changing the conditions so they are favourable to transposase activity. This minimizes the occurrence of off-target effects mediated by unbound transposase. Unbound transposase may be removed by isolating the target polynucleotide target polynucleotide in complex with the bound PGEP by an affinity purification technique that targets either the target polynucleotide or PGEP. Alternatively, the affinity purification technique may isolate the transposase not bound to the PGEP.

Conditions that are favourable to transposase activity are known in the art. Exemplary conditions that are favourable to transposase activity include those which comprise a salt concentration that is at least 50 mM but less than 250 mM. The conditions favourable to transposase activity may, for example, comprise a salt concentration of 50 to 250 mM, 60 to 240 mM, 70 to 230 mM, 80 to 220 mM, 90 to 210 mM, 100 to 200 mM, 110 to 190 mM, 120 to 180 mM, 130 to 170 mM, 140 to 160 mM, or 150 mM. Preferably, salt concentrations are at least 75 mM and less than 150 mM. Favourable transposase activity may be achieved at salt concentrations of 100 mM. Further exemplary conditions that are favourable to transposase activity include those which comprise the absence of a metal ion chelation agent and/or the presence of free Mg' ions. Conditions that are favourable to transposase activity may also reduce the transposase's capacity to bind the PGEP and/or capacity to bind DNA.

iv. Kinetic Inhibition

In the context of the present invention, the PGEP and transposase may have different kinetics in terms of the rate at which they will contact a target polynucleotide during a reaction. For example, a PGEP may be relatively slow to contact a target polynucleotide at a given region of interest — as determined by the polynucleotide sequence of its guide polynucleotide — whereas, conversely, a transposase may contact and react with a target polynucleotide relatively quickly.

The skilled person may therefore modulate the kinetics of the PGEP and/or the transposase in a method of the present invention in order to prevent the transposase contacting the target polynucleotide outside of any region of interest; and/or to prevent transposase-mediated insertion of one or more transposable elements into the target polynucleotide by the transposase outside of any given region of interest. Particularly, the kinetic activity of the transposase may be inhibited.

Examples of kinetic inhibition may involve sequential contacting of the target polynucleotide with the PGEP and the transposase such that the PGEP is allowed sufficient reaction time with the target polynucleotide to ensure all of the PGEP target sites are bound by PGEP prior to contacting the target polynucleotide with the transposase. This example may further involve contacting the target polynucleotide with the transposase such that the transposase is in stoichiometric balance with respect to the PGEP and/or the concentration of target regions of interest.

Examples of kinetic inhibition may also involve the application of transposase to the present method that has similar kinetic activity to the PGEP being used. This may be achievable by utilization of a naturally occurring transposase that has a similar kinetic activity to the PGEP being used, or this may be achievable by introducing mutations to a particular transposase, e.g. in its DNA-binding domain, in order to artificially reduce its kinetic activity.

### Systems

The present invention provides a system for preparing a nucleic acid construct suitable for single molecule characterization.

The invention provides a system comprising:
a polynucleotide-guided effector protein;
a guide polynucleotide binding domain;
a transposase; and
a transposable element comprising a modified polynucleotide,
   wherein the polynucleotide-guided effector protein directs said transposase to a region of interest within the target polynucleotide and further wherein the transposase inserts the transposable element into the polynucleotide, thereby producing a nucleic acid construct for single molecule characterisation.

Also provided is a system for preparing a nucleic acid construct, comprising:
a polynucleotide-guided effector protein;
a guide polynucleotide;
a transposase; and
a transposable element,

wherein the polynucleotide-guided effector protein and transposase are genetically fused or connected via a linker moiety, such that the transposase is directed to a region of interest within the target polynucleotide and inserts the transposable element into the target polynucleotide, thereby preparing a nucleic acid construct.

PGEPs, guide polynucleotides, transposases, transposable elements and modified polynucleotides are described in detail above. Any aspects described with respect to the method described herein for preparing a nucleic acid construct for single molecule characterization may also apply to the system for preparing a nucleic acid construct suitable for single molecule characterization

Methods of Detection and/or Characterization

The invention provides a is a method of detecting and/or characterising a target polynucleotide in a sample, comprising:
preparing a nucleic acid construct for single molecule characterisation according to the methods of preparing a nucleic acid construct described herein;
contacting the nucleic acid construct with a membrane comprising a transmembrane pore;
applying a potential difference across the membrane; and taking one or more measurements resulting from the contacting of the nucleic acid construct with the pore thereby detecting and/or characterising the target polynucleotide to determine the presence or absence of the target polynucleotide and/or one or more characteristics of the target polynucleotide.

Nucleic acid constructs prepared by the methods of the invention described herein may subsequently be applied to nanopore-based method of detecting and/or characterizing a target polynucleotide following the preparation of said nucleic acid construct from said target polynucleotide. Nanopore-based methods of detecting a target polynucleotide in a sample have been described previously (WO 2018/060740). Nanopore-based methods for characterising a target polynucleotide in a sample have been described previously (WO 2015/124935).

In the method of detecting a target polynucleotide described herein, prior to the step b. the method may comprise contacting the sample with a guide polynucleotide that binds to a sequence in the target polynucleotide and a polynucleotide-guided effector protein, wherein the guide polynucleotide and the polynucleotide-guided effector protein form a complex with any target in the sample.

In the method of detecting a target polynucleotide described herein, step (d) may further comprise monitoring for the presence or absence of an effect on the potential difference being applied across the membrane as a result of the interaction of the complex with the transmembrane pore, thereby determining the presence or absence of the target polynucleotide. The effect is indicative of the complex formed by the guide polynucleotide, the polynucleotide-guided effector protein and the nucleic acid construct interacting with the transmembrane pore. The effect may be caused by the translocation through the pore of an adaptor attached to one of the components of the complex, the target polynucleotide or the guide polynucleotide. The effect is indicative of the translocation through the pore of an adaptor attached to one of the components of the complex, the nucleic acid construct or the guide polynucleotide. The effect may be monitored using an electrical measurement and/or an optical measurement. In this case, the effect is a measured change or measured changes in an electrical or optical quantity. The electrical measurement may be a current measurement, an impedance measurement, a tunnelling measurement or a field effect transistor (FET) measurement. The effect may be a change in ion flow through the transmembrane pore resulting in a change in current, resistance or a change in an optical property. The effect may be electron tunneling across the transmembrane pore. The effect may be a change in potential due to the interaction of the complex with the transmembrane pore wherein the effect is monitored using localized potential sensor in a FET measurement.

In the method of characterising a target polynucleotide described herein, the contacting of the nucleic acid construct with the pore is such that at least one nucleic acid strand of the nucleic acid construct moves through the pore.

In the method of characterising a target polynucleotide described herein, the taking of one or more measurements are indicative of one or more characteristics of the target polynucleotide are selected from (i) the length of the polynucleotide, (ii) the identity of the polynucleotide, (iii) the sequence of the polynucleotide, (iv) the secondary structure of the polynucleotide and (v) whether or not the polynucleotide is modified.

Step b. comprises contacting the modified polynucleotide with a transmembrane pore such that the modified polynucleotide moves through the pore. The modified polynucleotide and the template polynucleotide may be contacted with a transmembrane pore such they both move through the pore.

Steps b. and c. of the method are preferably carried out with a potential applied across the pore. The applied potential may be a voltage potential. Alternatively, the applied potential may be a chemical potential. An example of this is using a salt gradient across an amphiphilic layer. A salt gradient is disclosed in Holden et al., J Am Chem Soc. 2007 Jul 11; 129(27):8650-5. In some instances, the current passing through the pore as the polynucleotide moves with respect to the pore is used to determine the sequence of the nucleic acid construct. This is strand sequencing. If the nucleic acid construct is sequenced, the sequence of the target polynucleotide may then be reconstructed.

The whole or only part of the nucleic acid construct and/or template polynucleotide may be characterized, for instance sequenced, using this method of characterising a target polynucleotide.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

Any transmembrane pore may be used in the invention. The pore may be biological or artificial. Suitable pores include, but are not limited to, protein pores, polynucleotide pores and solid state pores. In any of the methods described herein, the pore may permit translocation of double-stranded polynucleotide and bound polynucleotide through the pore. In any of the methods described herein, the pore may permit translocation of double-stranded polynucleotide and bound polynucleotide through the pore. In any of the methods described herein, the pore may permit translocation of a double-stranded polynucleotide. In any of the methods described herein, the pore may permit translocation of a single-stranded polynucleotide.

Any membrane may be used in accordance with the invention. Suitable membranes are well-known in the art. The membrane is preferably an amphiphilic layer. An amphiphilic layer is a layer formed from amphiphilic molecules, such as phospholipids, which have both at least one hydrophilic portion and at least one lipophilic or hydrophobic portion. The amphiphilic layer may be a monolayer or a bilayer. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphipiles. The copolymer may be a triblock, tetrablock or pentablock copolymer.

The amphiphilic layer is typically a planar lipid bilayer or a supported bilayer.

The amphiphilic layer is typically a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Suitable methods are disclosed in the Example. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

The lipid bilayer may be formed as described in International Application No. PCT/GB08/004127 (published as WO 2009/077734).

The membrane may be a solid state layer. A solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si3N4, Al2O3, and SiO, organic and inorganic polymers such as polyamide, plastics such as Teflon® or elastomers such as two-component addition-cure silicone rubber, and glasses. The solid state layer may be formed from monatomic layers, such as graphene, or layers that are only a few atoms thick. Suitable graphene layers are disclosed in International Application No. PCT/US2008/010637 (published as WO 2009/035647).

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial lipid bilayer. The layer may comprise other transmembrane and/or intramembrane proteins as well as other molecules in addition to the pore. Suitable apparatus and conditions are discussed below. The method of the invention is typically carried out in vitro.

The nucleic acid construct may be coupled to the membrane. This may be done using any known method. Particularly, the nucleic acid construct may be coupled to the membrane via a suitable modified polynucleotide ligated to the fragmented target polynucleotide. Alternatively, the modified polynucleotide ligated to the fragmented target polynucleotide may be modified in order to introduce a coupling or anchor element for coupling the nucleic acid construct to a membrane. If the membrane is an amphiphilic layer, such as a lipid bilayer (as discussed in detail above), the nucleic acid construct is preferably coupled to the membrane via a polypeptide present in the membrane or a hydrophobic anchor present in the membrane. The hydrophobic anchor is preferably a lipid, fatty acid, sterol, carbon nanotube or amino acid.

The nucleic acid construct may be coupled directly to the membrane. The polynucleotide is preferably coupled to the membrane via a linker. Preferred linkers include, but are not limited to, polymers, such as polynucleotides, polyethylene glycols (PEGs) and polypeptides. If a polynucleotide is coupled directly to the membrane, then some data will be lost as the characterising run cannot continue to the end of the nucleic acid construct due to the distance between the membrane and the pore. If a linker is used, then the polynucleotide can be processed to completion. If a linker is used, the linker may be attached to the polynucleotide at any position. The linker is preferably attached to the nucleic acid construct at the tail polymer.

The coupling may be stable or transient. For certain applications, the transient nature of the coupling is preferred. If a stable coupling molecule were attached directly to either the 5′ or 3′ end of a polynucleotide, then some data will be lost as the characterising run cannot continue to the end of the polynucleotide due to the distance between the bilayer and the pore. If the coupling is transient, then when the coupled end randomly becomes free of the bilayer, then the polynucleotide can be processed to completion. Chemical groups that form stable or transient links with the membrane are discussed in more detail below. The polynucleotide may be transiently coupled to an amphiphilic layer, such as a lipid bilayer using cholesterol or a fatty acyl chain. Any fatty acyl chain having a length of from 6 to 30 carbon atoms, such as hexadecanoic acid, may be used.

Suitable methods of coupling are disclosed in International Application No. PCT/GB12/05119 1 (published as WO 2012/164270) and UK Application No. 1406155.0.

A common technique for the amplification of sections of genomic DNA is using polymerase chain reaction (PCR). Here, using two synthetic oligonucleotide primers, a number of copies of the same section of DNA can be generated, where for each copy the 5′ of each strand in the duplex will be a synthetic polynucleotide. By using an antisense primer that has a reactive group, such as a cholesterol, thiol, biotin or lipid, each copy of the amplified target DNA will contain a reactive group for coupling.

The transmembrane pore is preferably a transmembrane protein pore. A transmembrane protein pore is a polypeptide or a collection of polypeptides that permits hydrated ions, such as analyte, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits analyte such as nucleotides to flow from one side of the membrane, such as a lipid bilayer, to the other. The transmembrane protein pore allows a polynucleotide, such as DNA or RNA, to be moved through the pore.

The transmembrane protein pore may be a monomer or an oligomer. The pore is preferably made up of several repeating subunits, such as 6, 7, 8 or 9 subunits. The pore is preferably a hexameric, heptameric, octameric or nonameric pore.

The transmembrane protein pore typically comprises a barrel or channel through which the ions may flow. The subunits of the pore typically surround a central axis and contribute strands to a transmembrane f3 barrel or channel or a transmembrane a-helix bundle or channel.

The barrel or channel of the transmembrane protein pore typically comprises amino acids that facilitate interaction with analytes, such as nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

The following Non-Limiting Examples Illustrate the Invention and are not Intended to be Limiting.

### EXAMPLE 1

This example demonstrates how two synthetic crRNA probes can be used to enrich for a region of a bacteriophage genome for nanopore sequencing. The enrichment occurs not by physical separation of target vs. non-target DNA, but by specifically inserting transposon adapters to the region of interest allowing specific sequencing starting within this region. Here is described a simple, one-pot approach, in which the enzymatic steps (dCas9 binding, adapter insertion by MuA, sequencing) are performed sequentially.

3.6kb lambda DNA (from SQK-LSK109) was end-repaired and dA-tailed using NEBNext® Ultra™ II End Repair/dA-Tailing Module (New England Biolabs, Inc., Cat # E7546L) according to the manufacturer's instructions. The mixture was then subjected to SPRI purification to remove contaminants and concentrate the DNA (AMPure XP beads, Beckman Coulter, Inc.) according to the manufacturer's instructions. The final library (“DCS”) was diluted to 100 ng/μL using Nuclease-free Water.

S. pyogenes Cas9 Nickase (D10A) ribonucleoprotein complexes (RNPs) were prepared as follows. Oligonucleotides AR369 (synthetic tracrRNA bearing 3′ DNA extension; TACATTTAAGACCCTAATAT/iSp18/[tracrRNA]) and pre-mixed AR147 (CTTCGCGGCAGATATAATGG) and AR148 (CCGACCACGCCAGCATATCG) (“crRNAs”-synthetic crRNAs mixed at a 1:1 equimolar ratio) were first annealed by incubating 1 μL of AR369 (at 100 μM), 1 μL crRNAs (at 100 μM) and 8 μL nuclease-free duplex buffer (Integrated DNA Technologies, Inc., Cat # 11-01-03-01) at 95° C. for 5 min, followed by cooling to room temperature to form 10 μM tracrRNA-crRNA complex. RNPs were then formed by incubating 2.5 μL of tracrRNA-crRNA complex (800 nM final concentration) with 400 nM S. pyogenes Cas9 (New England Biolabs, Inc., Cat # M0650T) in a total of 30 μL NEB CutSmart buffer at room temperature for 30 mins. This step yielded 30 μL of “Cas9 RNPs”.

MuA Transposase ribonucleoprotein complexes (RNPs) were prepared as follows. Oligonucleotides EN47 (MuA bottom strand bearing 3′ DNA extension annealing to the tracrRNA) and EN45 (/DBCO-TEG/GCTTGGGTGTTTAACCGTTTTCGCATTTATCGTGAAACGCTTTCGCGTTTT TCGTGCGCCGCTTCA) were first annealed by incubating 10 μL of EN47 (5-GATCTGAAGCGGCGCACGAAAAACGCGAAAGCGTTTCACGATAAATGCGAAA ACTTTTTTTTTTATATTAGGGTCTTAAATGTA; at 100 μM), 10 μL EN45 (at 100 μM) and 5 μL nuclease-free duplex buffer (Integrated DNA Technologies, Inc., Cat # 11-01-03-01) at 95° C. for 5 min, followed by cooling to room temperature to form 10 μM

MuA Y-adapter. RNPs were then formed by incubating 5 μL of MuA Y-adapter complex (8 μM final concentration) with 3.3 μM MuA transposase (purified in-house by Oxford Nanopore Technologies) in a total of 25 μL MuA reaction buffer at 30° C. for 60 mins. This step yielded 25 μL of “MuA RNPs”.

Three Distinct Reactions were Performed in Four Single Tubes as follows:

(1) A reaction in which rapid sequencing adapter was clicked to MuA Y-adapter transposed DNA, wherein Cas9 RNPs were added to the reaction mix,

100 ng of DCS was bound by Cas9 RNPs by incubation of 1 μL library, 5 μL Cas9 RNPs (above), 2.5 μL NEB CutSmart Buffer, 22.5 μL nuclease-free water for a total of 25 μL. This mixture was incubated at 37° C. for 10 mins to bind Cas9 to the target regions, then 20 mM of EDTA, 50 μM of TCEP and 150 mM NaCl were added. The mixture was incubated for further 10 mins at 37° C. This step yielded 100 ng “target DNA, bound by Cas9 RNPs”.

(2) A reaction in which rapid sequencing adapter was clicked to MuA Y-adapter transposed DNA, wherein MuA RNPs were added to the reaction mix,

100 ng of DCS or 1 μL of library was added to 2.5 μL NEB CutSmart Buffer, 27.5 μL nuclease-free water for a total of 25 μL. This mixture was incubated at 37° C. for 10 mins, then 100 nM of MuA RNPs, 20 mM of EDTA, 50 μM of TCEP and 150 mM NaCl were added. The mixture was incubated for further 10 mins at 37° C. This step yielded 100 ng “target DNA, bound by MuA RNPs”.

(3) A reaction in which rapid sequencing adapter was clicked to MuA Y-adapter transposed DNA, wherein Cas9 RNPs and MuA RNPs were added sequentially to the reaction mix,

100 ng of DCS was bound by Cas9 RNPs by incubation of 1 μL library, 5 μL Cas9 RNPs (above), 2.5 μL NEB CutSmart Buffer, 22.5 μL nuclease-free water for a total of 25 μL. This mixture was incubated at 37° C. for 10 mins to bind Cas9 to the target regions, then 100 nM of MuA RNPs, 20 mM of EDTA, 50 μM of TCEP. The mixture was incubated for further 10 mins at 37° C. This step yielded 100 ng “target DNA, bound by MuA and Cas9 RNPs”.

(4) A reaction in which rapid sequencing adapter was clicked to MuA Y-adapter transposed DNA, wherein Cas9 RNPs and MuA RNPs were added sequentially to the reaction mix and the salt concentration was increased of 150mM NaCl,

100 ng of DCS was bound by Cas9 RNPs by incubation of 1 μL library, 5 μL Cas9 RNPs (above), 2.5 μL NEB CutSmart Buffer, 22.5 μL nuclease-free water for a total of 25 μL. This mixture was incubated at 37° C. for 10 mins to bind Cas9 to the target regions, then 100 nM of MuA RNPs, 20 mM of EDTA, 50 μM of TCEP and 150 mM NaCl were added. The mixture was incubated for further 10 mins at 37° C. This step yielded 100 ng “target DNA, bound by MuA and Cas9 RNPs in higher salt”.

The mixtures were then subjected to SPRI purification to remove unligated adapter and other contaminants. 2 volumes (˜50 μL) SPRI beads (AMPure XP beads, Beckman Coulter, Inc.) were added to adapter-ligated DNA, mixed gently by inversion, and incubated for 10 min at room temperature to bind the adapter-ligated DNA to the beads.

The beads were pelleted using a magnetic separator, the supernatant removed, and washed twice with 250 μL SFB (from Oxford Nanopore LSK-109), with complete resuspension of the beads at each wash and repelleting of the beads following the wash. Following the second wash, the beads were pelleted once more, the excess wash buffer removed, and the DNA eluted from the beads by resuspension of the bead pellet in 13.5 μL Tris elution buffer (10 mM Tris-Cl, 20 mM NaCl, pH 7.5 at room temperature) for 10 min at room temperature. The beads were pelleted once more and the eluate (supernatant), containing purified gDNA, adapted at the target sites, retained. The DNA cleavage and transposon adapter insertion were initiated by the addition of 1.5 μL of Cutsmart buffer (New England Biolabs, Inc., Cat # B7204S) and incubating the mixture for 2 mins at 30° C. and 2 mins at 80° C.

Sequencing adapter (“RAP” from SQK-RAD004) was ligated to the DNA strands via click chemistry. 1 μL of RAP was added to the mixture and incubate for 10 mins at room temperature.

37.5 μL SQB and 25.5 μL LB (both from Oxford Nanopore Technologies' LSK-108) were added to 15 μL of the eluate to yield “MinION sequencing mix” for a final volume of 75 μL.

To sequence target DNA, an Oxford Nanopore Technologies FLO-MIN106 flowcell was prepared by introducing 800 μL flowcell preparation mix (prepared using: 1170 μL FLB and 30 μL of FLT from Oxford Nanopore LSK-109) via the inlet port. The SpotON port was subsequently opened and a further 200 μL flowcell preparation mix perfused via the inlet port. 75 μL of MinION sequencing mix were added to the flowcell via the SpotON port, and the ports closed. 6 h of sequencing data were collected using Oxford Nanopore Technologies' MinKNOW (version 19.06.8), and subsequently basecalled (using Guppy) and aligned to the 3.6kb lambda reference genome offline.

### Results

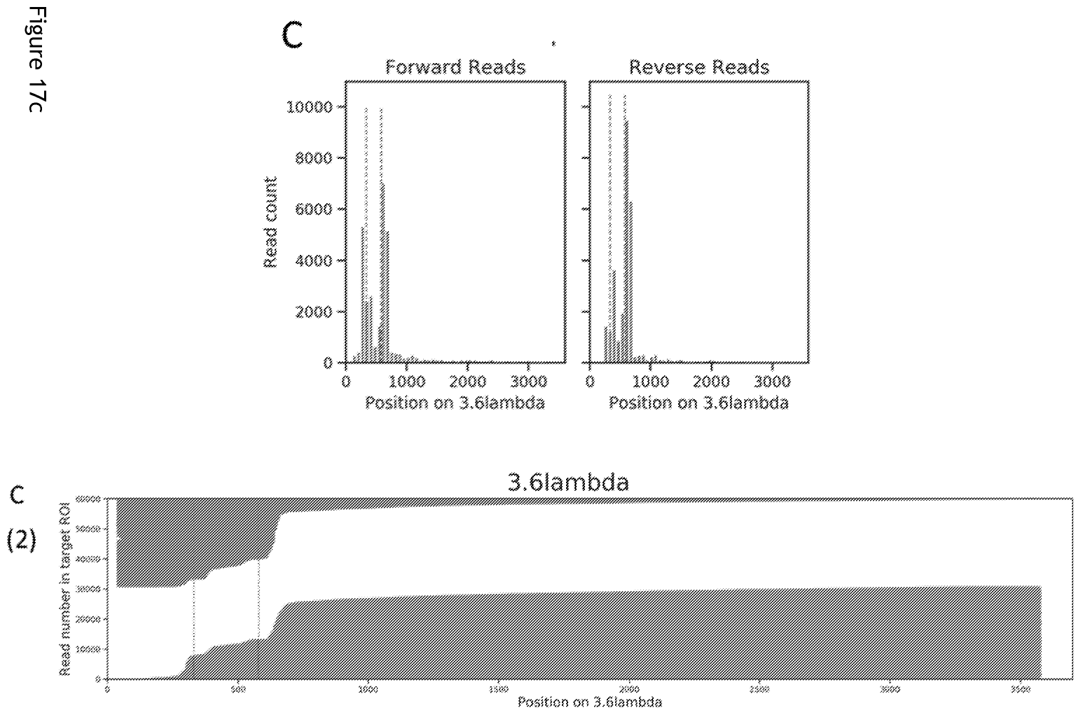

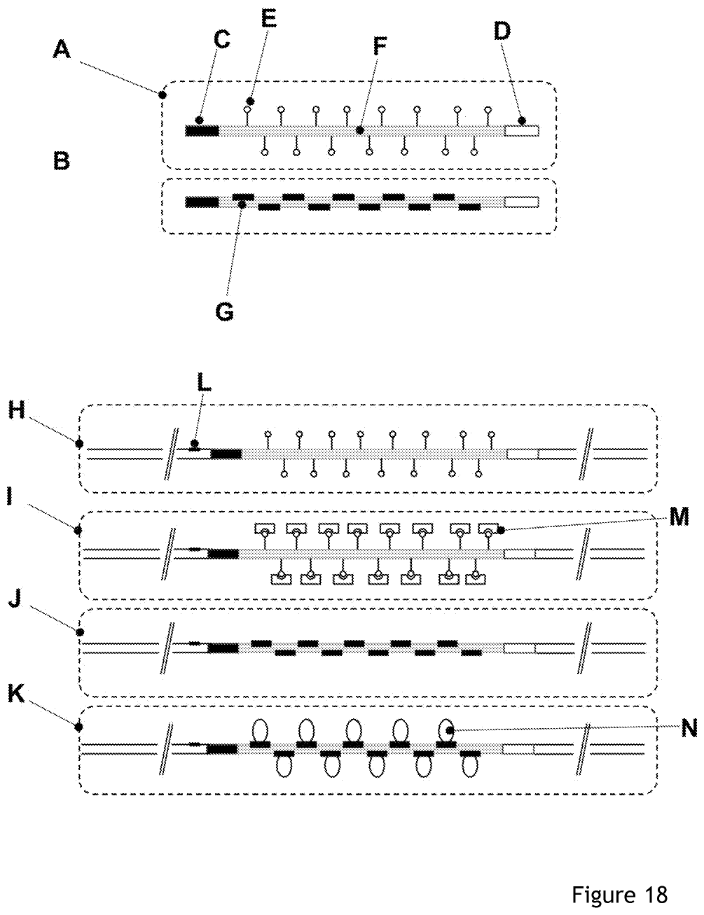

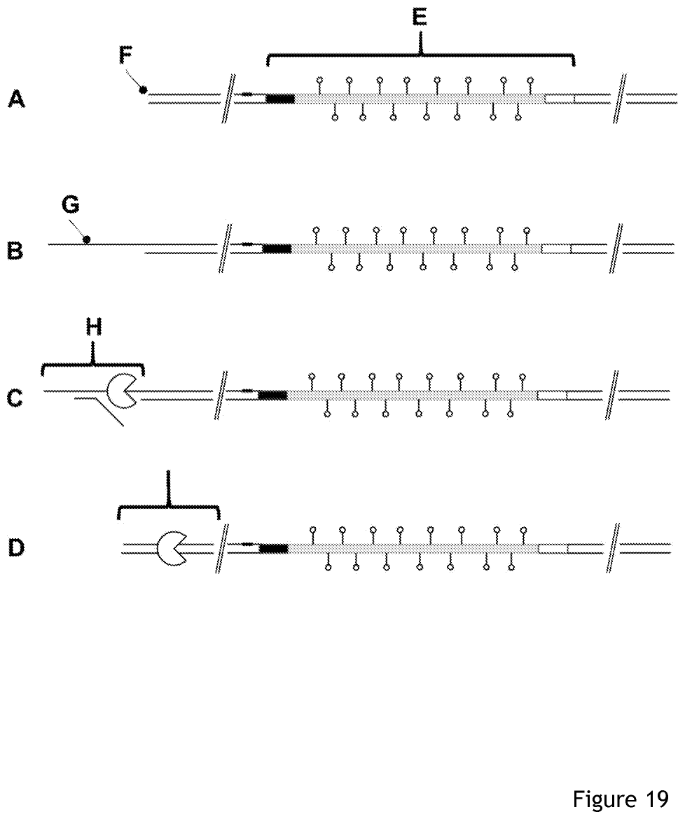

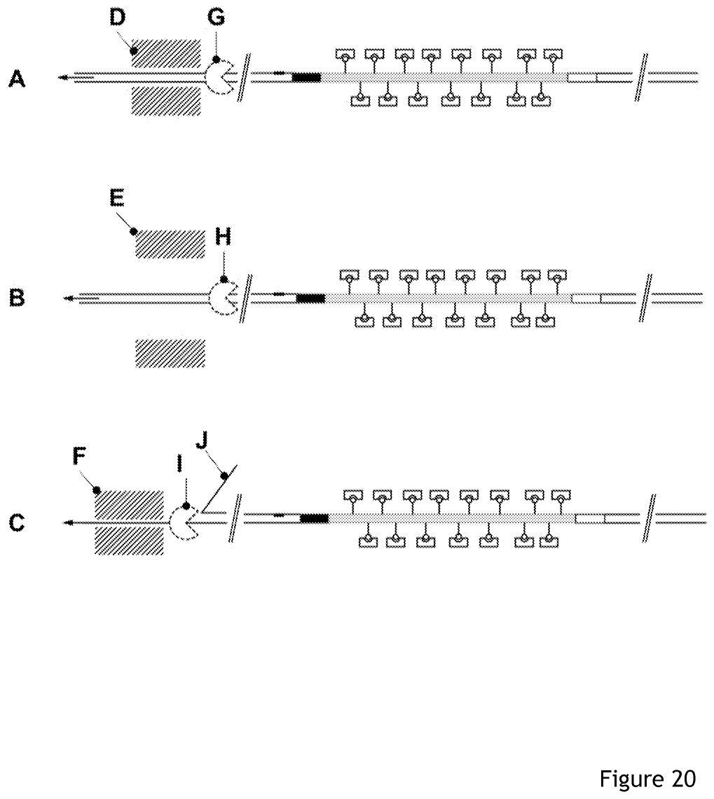

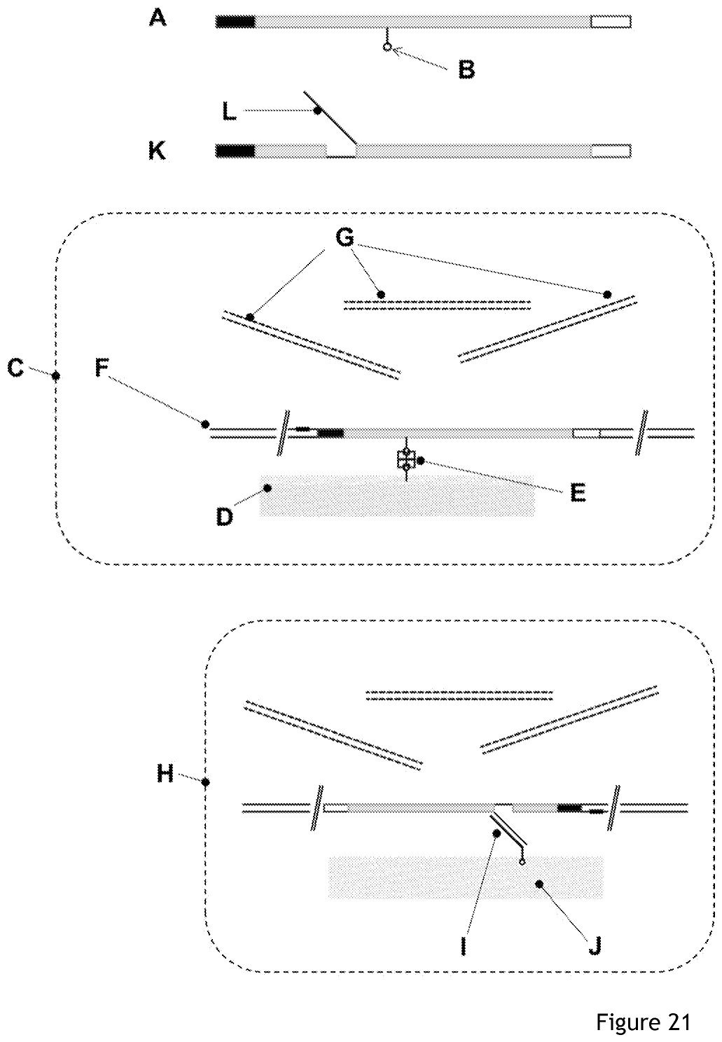

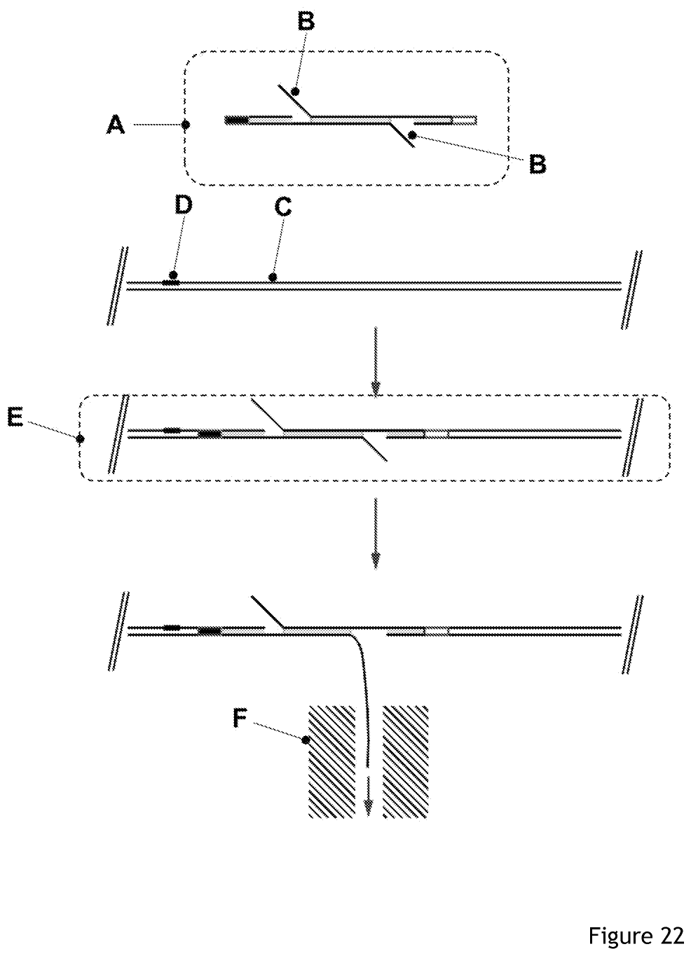

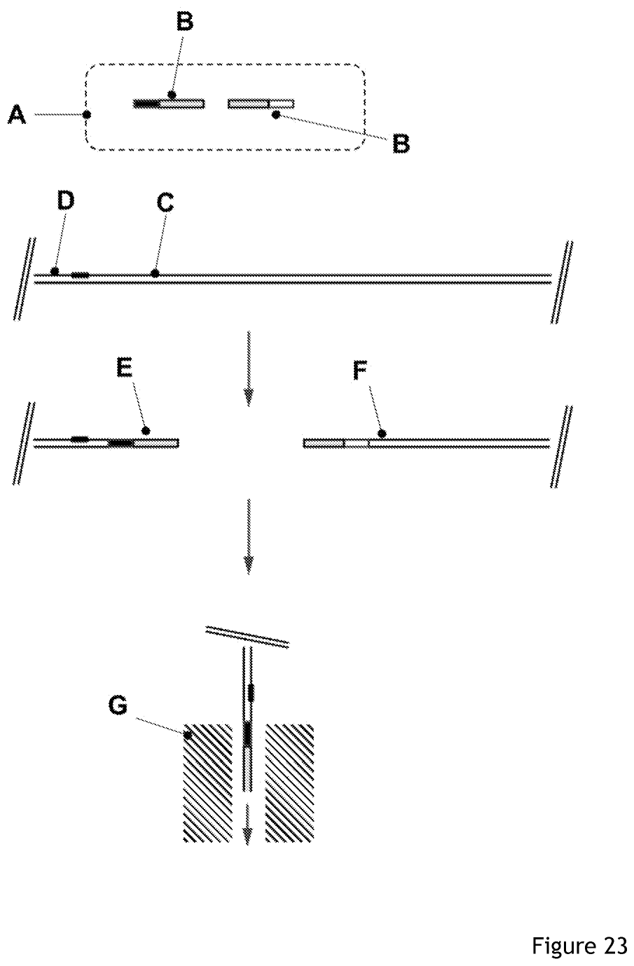

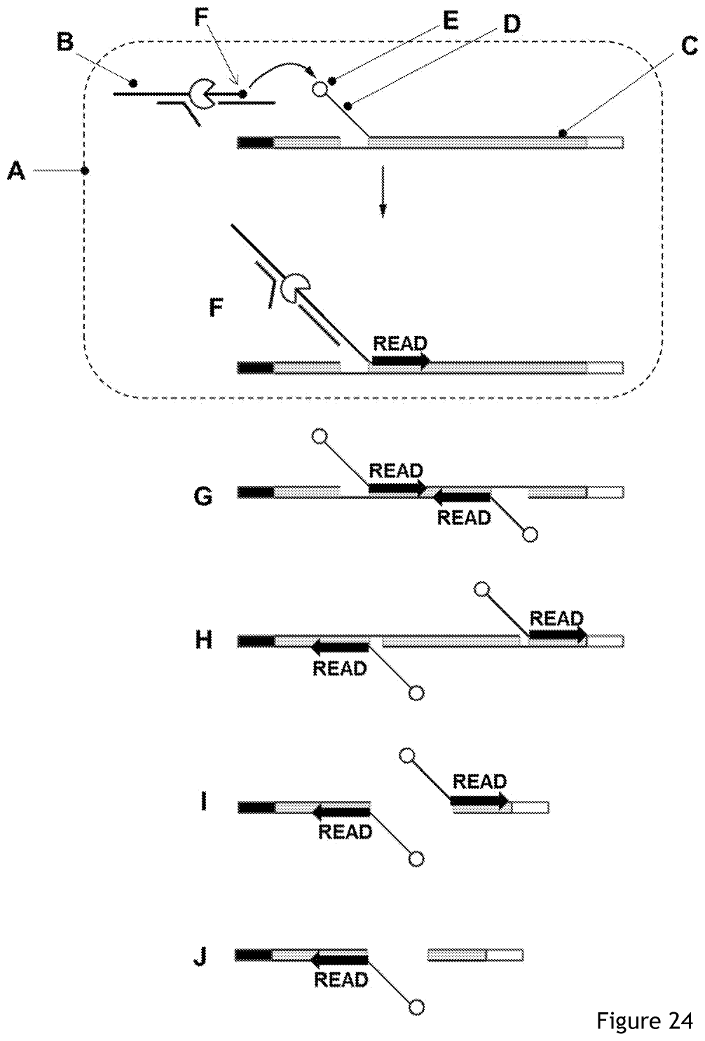

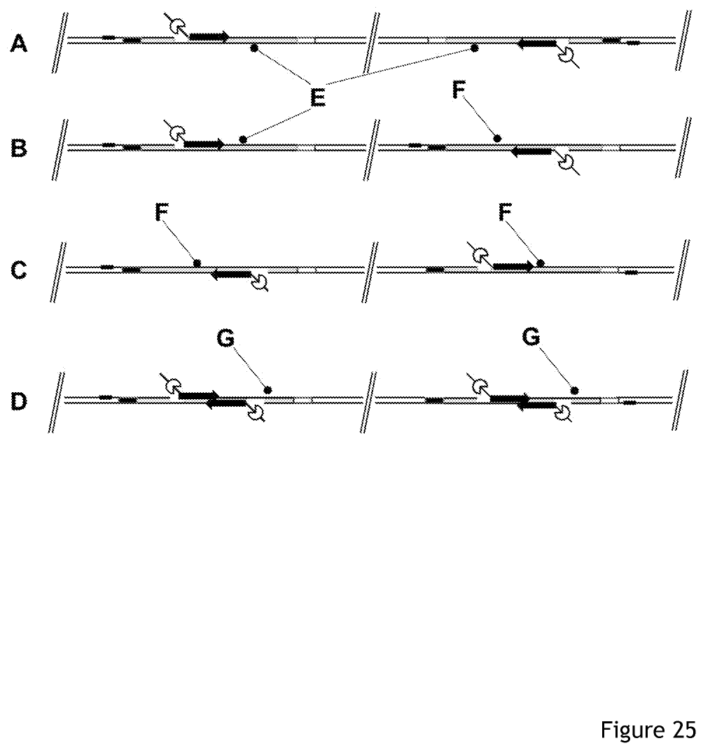

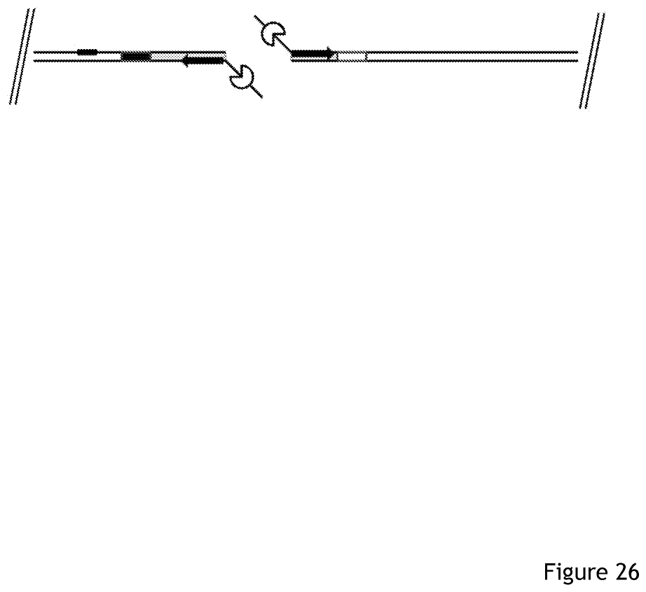

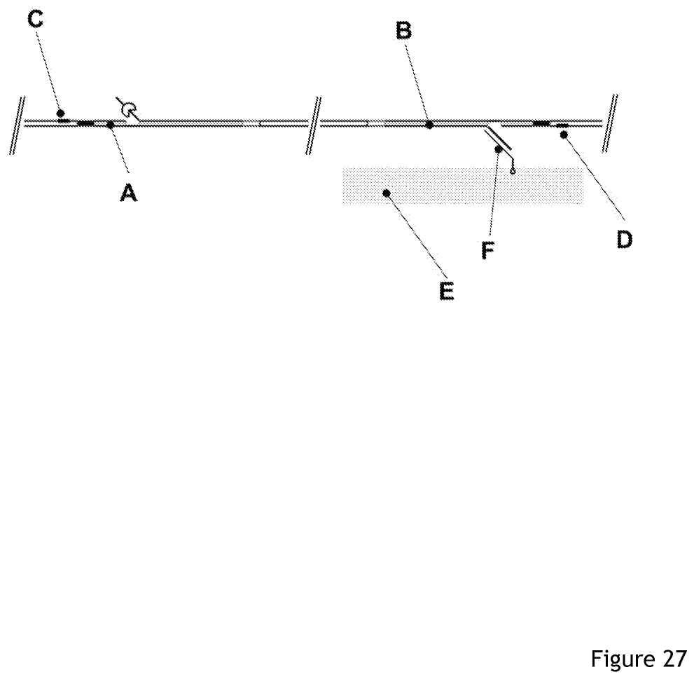

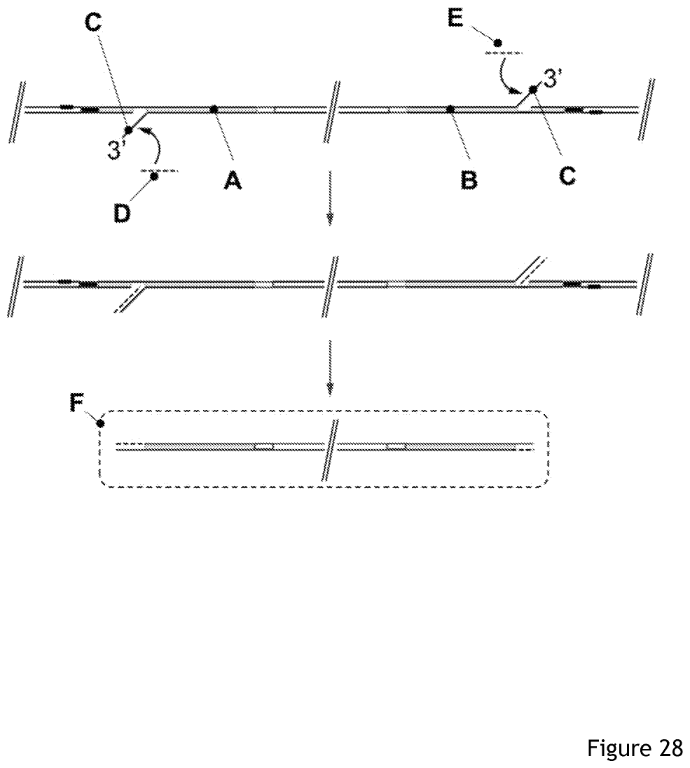

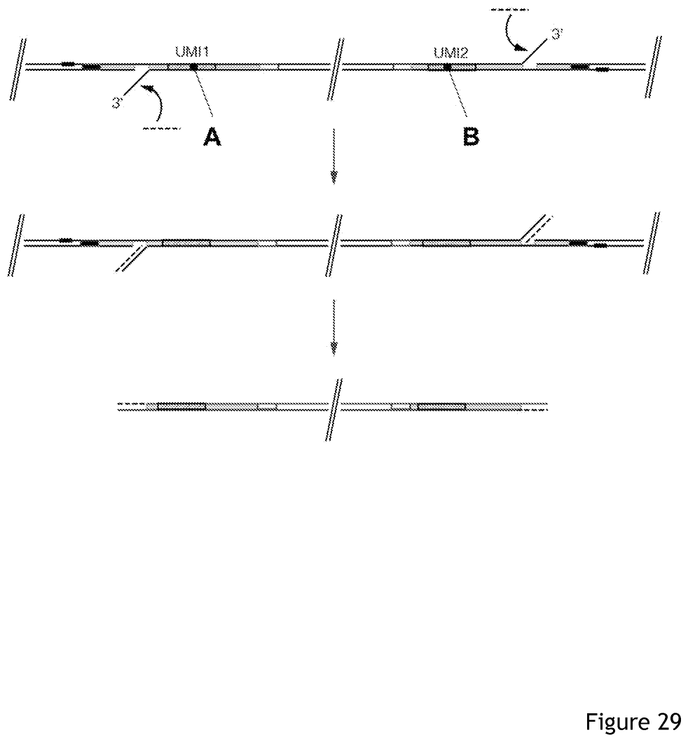

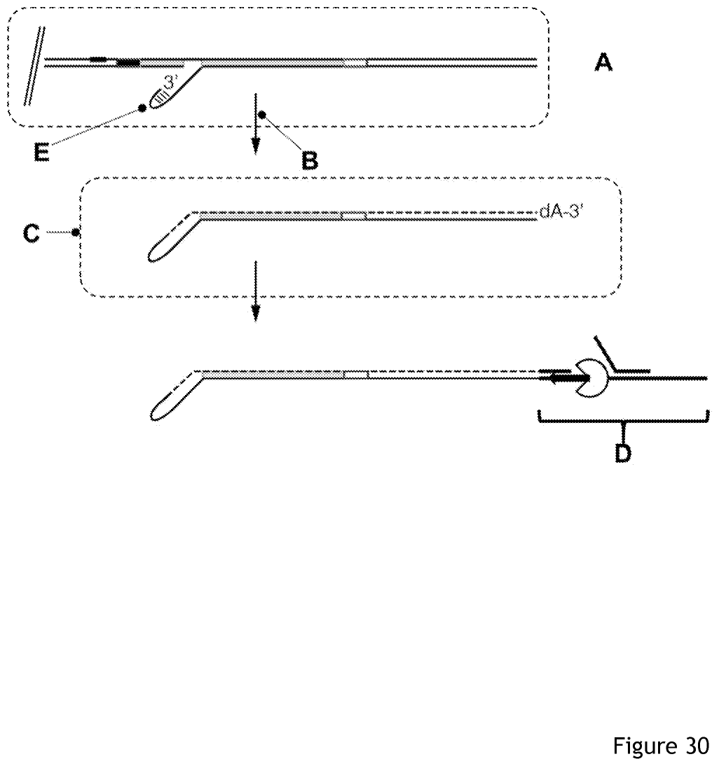

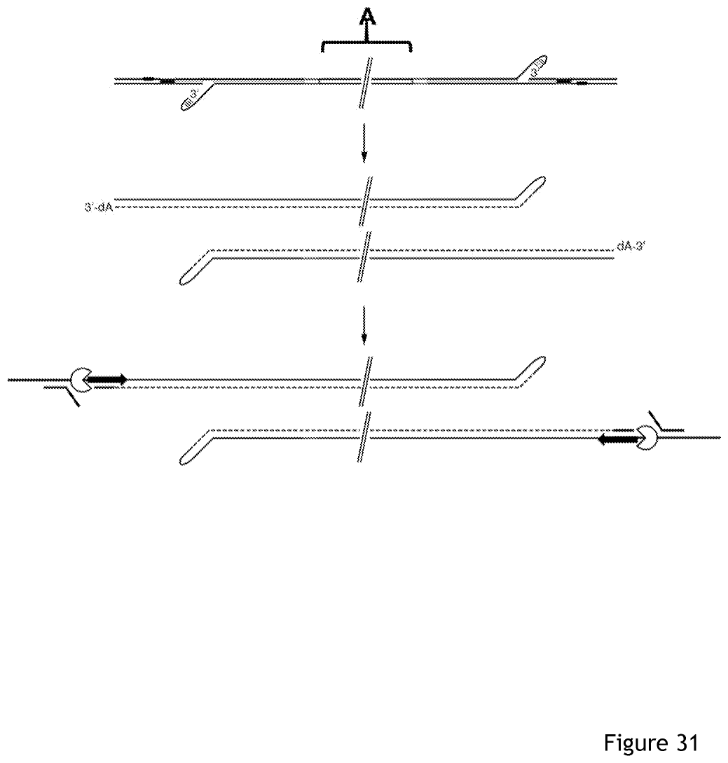

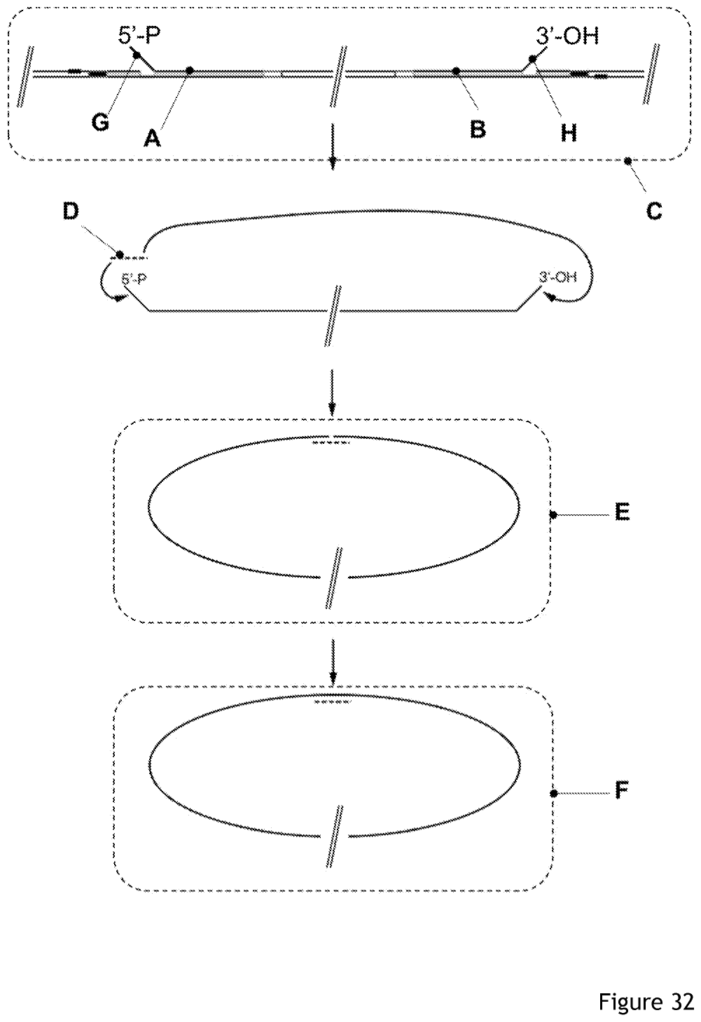

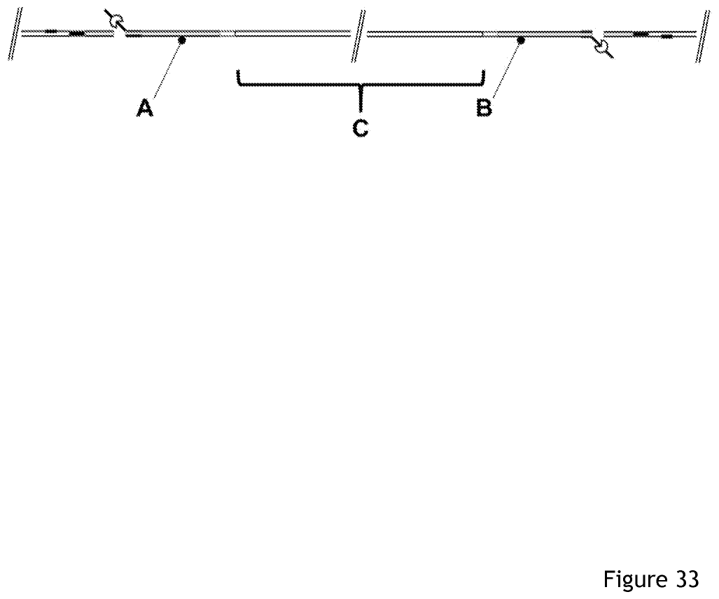

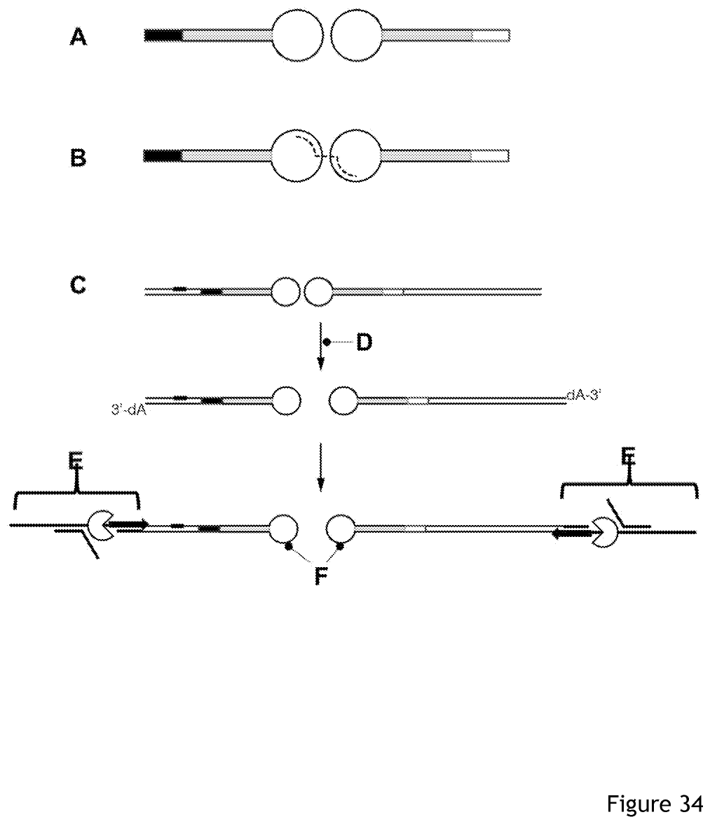

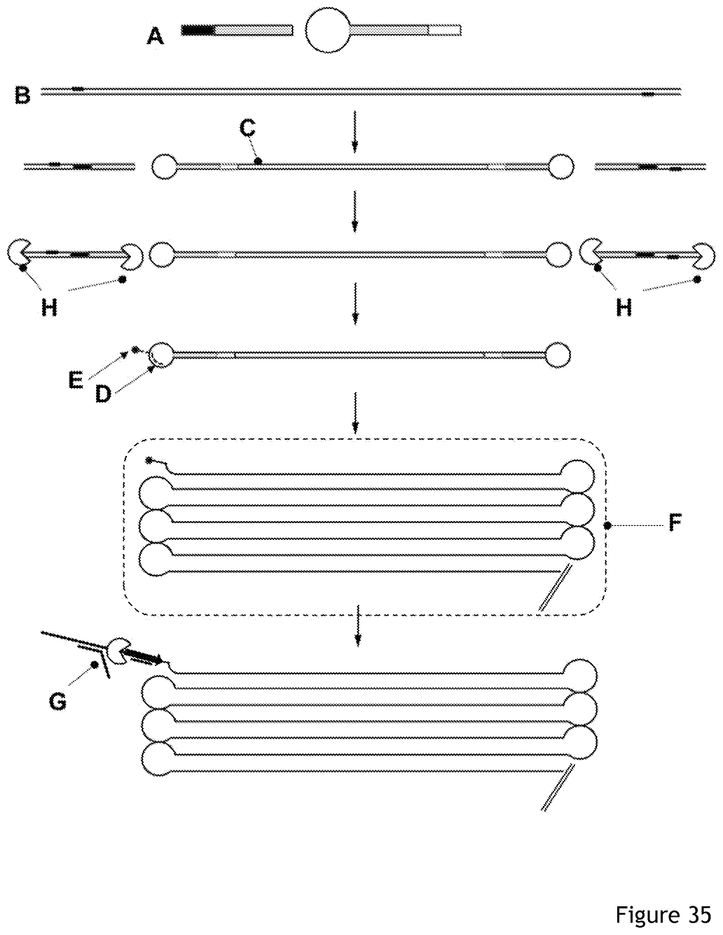

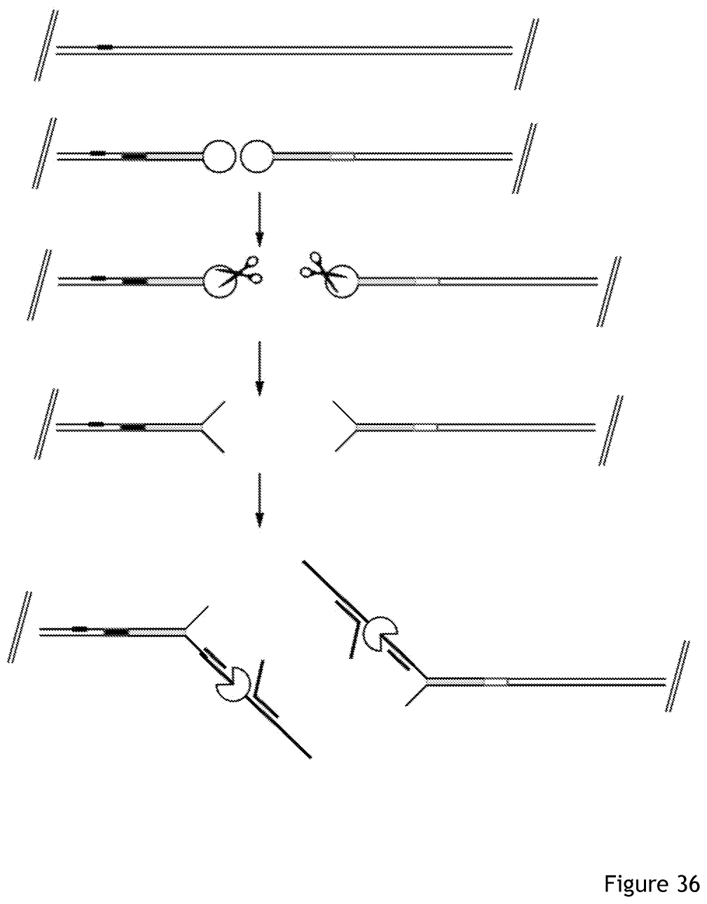

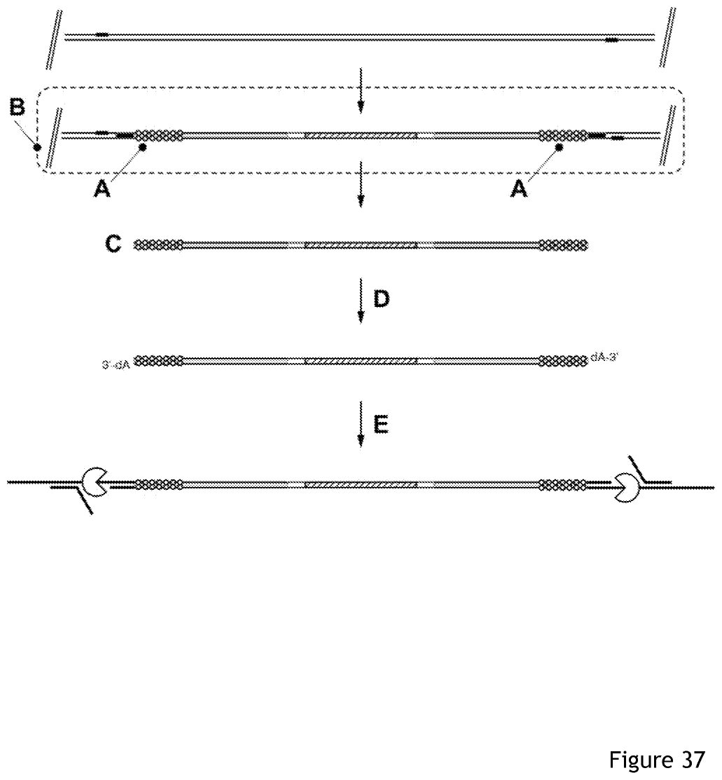

FIG. 17 and panel 1 shows the start of the reads relative to the 3.6kb lambda reference resulting from alignment of sequencing reads to the relative to 3.6kb lambda reference. Enrichment of the target read starts was observed in conditions (3) and (4), as expected, showing that MuA cuts predominantly in the correct location within a 150nt window around dCas9 binding sites and that, the adapted cut sites were efficiently clicked to the sequencing adapter.

FIG. 17 and panel 2 shows the pileups resulting from alignment of sequencing reads to the 3.6kb lambda reference. Enrichment of the target regions was observed in conditions (3) and (4), as expected, showing that MuA cut predominantly in the correct location. Approximately 80% of all reads mapped started in a 150bp window around the predicted dCas9 binding sites.

### EXAMPLE 2

### Lambda—Cas12k-Transposon Sequential

This example demonstrates how a single synthetic crRNA probes can be used to enrich for a region of a bacteriophage genome for nanopore sequencing. The enrichment occurs not by physical separation of target vs. non-target DNA, but by specifically inserting a transposon cargo to the region of interest allowing specific sequencing starting within the inserted cargo. Here is described a simple, one-pot approach, in which the enzymatic steps (dCas9 binding, adapter insertion by MuA, sequencing) are performed sequentially.

### Materials and Methods

3.6kb lambda DNA (from SQK-LSK109) was end-repaired and dA-tailed using NEBNext® Ultra™ II End Repair/dA-Tailing Module (New England Biolabs, Inc., Cat # E7546L) according to the manufacturer's instructions. The mixture was then subjected to SPRI purification to remove contaminants and concentrate the DNA (AMPure XP beads, Beckman Coulter, Inc.) according to the manufacturer's instructions. The final library (“DCS”) was diluted to 100 ng/μL using Nuclease-free Water.

Transposon ribonucleoprotein complexes (RNPs) were prepared as follows. Oligonucleotides ARXX (synthetic tracrRNA) and ARXX (“crRNAs”-synthetic crRNA) were first annealed by incubating 1 μL of ARXX (at 100 μM), 1 μL cRNAs (at 100 μM) and 8 μL nuclease-free duplex buffer (Integrated DNA Technologies, Inc., Cat # 11-01-03-01) at 95° C. for 5 min, followed by cooling to room temperature to form 10 μM tracrRNA-crRNA complex. RNPs were then formed by incubating 2.5 μL of tracrRNA-crRNA complex (800 nM final concentration) with 800nM of ARXX (“Cargo”—dsDNA bearing the transposon recognition sites and adapter insert) 400 nM of each transposon proteins (Cas12k, TniQ, TnsB and TnsC) in a total of 30 μL NEB CutSmart buffer at room temperature for 60 mins. This step yielded 30 μL of “Transposon RNPs”. Three distinct reactions were performed in four single tubes as follows:
(1) A reaction in which rapid sequencing adapter was clicked to adapter transposed DNA, wherein Cas12k was omitted from the Transposon RNPs which were added to the reaction mix,

100 ng of DCS was transposed by Transposon RNPs by incubation of 1 μL library, 5 μL Transposon RNPs (above but omitting Cas12k), 2.5 μL NEB CutSmart Buffer, 22.5 μL nuclease-free water for a total of 25 μL. This mixture was incubated at 37° C. for 20 mins to bind Cas12k to the target regions This step yielded 100 ng “target DNA, bound by Transposon RNPs (-Cas12k)”.
(2) A reaction in which rapid sequencing adapter was clicked to adapter transposed DNA, wherein TniQ was omitted from the Transposon RNPs which were added to the reaction mix,

100 ng of DCS was transposed by Transposon RNPs by incubation of 1 μL library, 5 μL Transposon RNPs (above but omitting TniQ), 2.5 μL NEB CutSmart Buffer, 22.5 μL nuclease-free water for a total of 25 μL. This mixture was incubated at 37° C. for 20 mins to bind Cas12k to the target regions This step yielded 100 ng “target DNA, bound by Transposon RNPs (-TniQ)”.
(3) A reaction in which rapid sequencing adapter was clicked to adapter transposed DNA, wherein the cargo was omitted from the Transposon RNPs which were added to the reaction mix,

100 ng of DCS was transposed by Transposon RNPs by incubation of 1 μL library, 5 μL Transposon RNPs (above but omitting the cargo DNA), 2.5 μL NEB CutSmart Buffer, 22.5 μL nuclease-free water for a total of 25 μL. This mixture was incubated at 37° C. for 20 mins to bind Cas12k to the target regions This step yielded 100 ng “target DNA, bound by Transposon RNPs (-cargo)”.
(4) A reaction in which rapid sequencing adapter was clicked to adapter transposed DNA, wherein the Transposon RNPs were added to the reaction mix,

100 ng of DCS was transposed by Transposon RNPs by incubation of 1 μL library, 5 μL Transposon RNPs (above), 2.5 μL NEB CutSmart Buffer, 22.5 μL nuclease-free water for a total of 25 μL. This mixture was incubated at 37° C. for 20 mins to bind Cas12k to the target regions This step yielded 100 ng “target DNA, bound by Transposon RNPs”.

Sequencing adapter (“RAP” from SQK-RAD004) was ligated to the DNA strands via click chemistry. 1 μL of RAP was added to the mixture and incubate for 10 mins at room temperature.

37.5 μL SQB and 12.5 μL LB (both from Oxford Nanopore Technologies' LSK-108) were added to 25 μL of the mixtures to yield “MinION sequencing mixes” for a final volume of 75 μL.

To sequence target DNA, an Oxford Nanopore Technologies FLO-MIN106 flowcell was prepared by introducing 800 μL flowcell preparation mix (prepared using: 1170 μL FLB and 30 μL of FLT from Oxford Nanopore LSK-109) via the inlet port. The SpotON port was subsequently opened and a further 200 μL flowcell preparation mix perfused via the inlet port. 75 μL of MinION sequencing mix were added to the flowcell via the SpotON port, and the ports closed. 6 h of sequencing data were collected using Oxford Nanopore Technologies' MinKNOW (version 19.06.8), and subsequently basecalled (using Guppy) and aligned to the 3.6kb lambda reference genome offline.

### Results

FIG. 17 shows the pileups resulting from alignment of sequencing reads to the 3.6kb lambda reference.

1-50. (canceled)

51. A method of preparing a nucleic acid construct for single molecule characterisation, comprising contacting a target polynucleotide with:
a polynucleotide-guided effector protein,
a guide polynucleotide;
a transposase; and
a transposable element comprising a modified polynucleotide,
wherein the polynucleotide-guided effector protein directs said transposase to a region of interest within the target polynucleotide and the transposase inserts the transposable element into the polynucleotide, thereby producing a nucleic acid construct for single molecule characterisation.

52. A method according to claim 51, wherein the polynucleotide-guided effector protein binds to the transposase via a protein-protein interaction.

53. A method according to claim 51, wherein the polynucleotide-guided effector protein is genetically fused to the transposase.

54. A method according to claim 51, wherein the polynucleotide-guided effector protein is connected to the transposase via a linker moiety.

55. A method of preparing a nucleic acid construct, comprising contacting a target polynucleotide with:
a polynucleotide-guided effector protein
a guide polynucleotide;
a transposase; and
a transposable element,
wherein the polynucleotide-guided effector protein and transposase are genetically fused or connected via a linker moiety such that the transposase is directed to a region of interest within the target polynucleotide and inserts the transposable element into the target polynucleotide, thereby preparing a nucleic acid construct.

56. A method according to claim 55, wherein the transposable element comprises a modified polynucleotide.

57. A method according to 51, wherein the linker moiety is a linker polynucleotide that binds to the polynucleotide-guided effector protein and to the transposase, optionally wherein the linker polynucleotide is a tracrRNA, a CRISPR RNA or a single guide RNA and wherein the linker moiety sequence length determines the length of the sequence between a protospacer adjacent motif (PAM) immediately upstream of the sequence within the target polynucleotide contacted by the polynucleotide-guided effector protein and the site in which the transposase inserts the transposable element into the region of interest.

58. A method according to claim 51, wherein the target polynucleotide is contacted with the polynucleotide-guided effector protein, the guide polynucleotide, the transposase and the transposasable element under conditions unfavourable to transposase activity, and following binding of the transposase to the polynucleotide-guided effector protein, wherein the method comprises changing the conditions so they are favourable to transposase activity.

59. A method according to claim 51, wherein the target polynucleotide is contacted with a complex comprising the polynucleotide-guided effector protein, the guide polynucleotide, the transposase and the transposable element.

60. A method according to claim 51, wherein the guide polynucleotide is a guide RNA and the polynucleotide-guided effector protein is a RNA-guided effector protein, optionally wherein the RNA-guided effector protein is a RNA-guided endonuclease or an RNA-guided endonuclease wherein the nuclease activity of the RNA-guided endonuclease is disabled.

61. A method according to claim 51, wherein the polynucleotide-guided effector protein is an assembly of multiple protein components, optionally wherein the polynucleotide-guided effector protein is (i)Cascade which comprises an assembly of Cas6-Cas7-Cas8 proteins or (ii)wherein the polynucleotide-guided effector protein comprises one or more components including Cas, Cpf 1 or C2c2 or (iii) is Cas12k.

62. A method according to claim 51, wherein the transposase is a multimeric protein and the multimeric protein comprises the maize Ac transposon, the Drosophila P element, Tn5, Tn7, Tn10, Mariner, IS10, IS50 or MuA.

63. A method according to claim 51, wherein the modified polynucleotide comprises a click reactive group, a fluorophore, a conjugation agent, a pull down group, a tethering moiety, a marker, a modified base, an abasic residue and/or a spacer and wherein the marker or pull down agent is biotin, and/or the modified polynucleotide comprises a base-base conjugate and/or a protein-base conjugate, and/or the tethering moiety is a polypeptide comprising a hydrophobic region, a lipid, fatty acid, sterol, carbon nanotube, polypeptide, protein or amino acid, cholesterol, palmitate or tocopherol, further wherein the transposable element is an adaptor, such as a sequencing adaptor, an intermediate adaptor, an amplification adaptor, a hairpin adaptor, a unique molecular identifier or a rolling circle amplification template .

64. A method of detecting and/or characterising a target polynucleotide in a sample, comprising:
(i) preparing a nucleic acid construct for single molecule characterisation according to the method of claim 51;
(ii) contacting the nucleic acid construct with a membrane comprising a transmembrane pore;
(iii) applying a potential difference across the membrane; and
(iv) taking one or more measurements resulting from the contacting of the nucleic acid construct with the pore thereby detecting and/or characterising the target polynucleotide to determine the presence or absence of the target polynucleotide and/or one or more characteristics of the target polynucleotide.

65. A method according to claim 64, wherein the one or more characteristics of the target polynucleotide are selected from (i) the length of the polynucleotide, (ii) the identity of the polynucleotide, (iii) the sequence of the polynucleotide, (iv) the secondary structure of the polynucleotide and (v) whether or not the polynucleotide is modified.

66. A method according to 55, wherein the linker moiety is a linker polynucleotide that binds to the polynucleotide-guided effector protein and to the transposase, optionally wherein the linker polynucleotide is a tracrRNA, a CRISPR RNA or a single guide RNA and wherein the linker moiety sequence length determines the length of the sequence between a protospacer adjacent motif (PAM) immediately upstream of the sequence within the target polynucleotide contacted by the polynucleotide-guided effector protein and the site in which the transposase inserts the transposable element into the region of interest.

67. A method according to 55, wherein the target polynucleotide is contacted with a complex comprising the polynucleotide-guided effector protein, the guide polynucleotide, the transposase and the transposable element.

68. A method according to 55, wherein the guide polynucleotide is a guide RNA and the polynucleotide-guided effector protein is a RNA-guided effector protein, optionally wherein the RNA-guided effector protein is a RNA-guided endonuclease or an RNA-guided endonuclease wherein the nuclease activity of the RNA-guided endonuclease is disabled.

69. A method according to 55, wherein
(i) the guide polynucleotide is a guide RNA and the polynucleotide-guided effector protein is a RNA-guided effector protein, optionally wherein the RNA-guided effector protein is a RNA-guided endonuclease or an RNA-guided endonuclease wherein the nuclease activity of the RNA-guided endonuclease is disabled; or
(ii) the transposase is a multimeric protein and the multimeric protein comprises the maize Ac transposon, the Drosophila P element, Tn5, Tn7, Tn10, Mariner, IS10, IS50 or MuA.

70. A method of detecting and/or characterising a target polynucleotide in a sample, comprising:
(i) preparing a nucleic acid construct for single molecule characterisation according to the method of claim 55;
(ii) contacting the nucleic acid construct with a membrane comprising a transmembrane pore;
(iii) applying a potential difference across the membrane; and
(iv) taking one or more measurements resulting from the contacting of the nucleic acid construct with the pore thereby detecting and/or characterising the target polynucleotide to determine the presence or absence of the target polynucleotide and/or one or more characteristics of the target polynucleotide.
